(19) United States
(12) Patent Application Publication
(10) Pub. No.: US 2007/0059454 A1
(43) Pub. Date: Mar. 15, 2007

(54) Products for treating and preventing chronic diseases: eliminating the autoimmune triggers that underly chronic disease

(76) Inventor: Karen M. Slimak, Springfield, VA (US)

Correspondence Address: STEVENS DAVIS MILLER & MOSHER, LLP, 1615 L STREET, NW, SUITE 850, WASHINGTON, DC, 20036, US

(21) Appl. No.: 10/557,357

(22) PCT Filed: Jan. 7, 2004
(86) PCT No.: PCT/US04/00224

§ 371 Date: Nov. 17, 2005

Related U.S. Application Data
(60) Provisional application No. 60/438,294, filed on Jan. 7, 2003.
(60) Provisional application No. 60/438,779, filed on Jan. 9, 2003.

(51) Int. Cl. B05D 7/00; C09J 103/00; A61K 9/00

(52) U.S. Cl. 427/421.1; 424/400; 106/125.1

(57) ABSTRACT

Long term exposure to volatile organic components (VOCs) has been linked to chronic diseases, conditions and symptoms such as diseases of the blood vessels, heart and/or lung: psychiatric disorders, diseases of the nervous system and genetic diseases, among others. Methods and products are disclosed to treat chronically ill patients as well as healthy patients to reduce or eliminate exposure to VOCs.

### RELATED PATENTS

The following related patents and other materials are hereby incorporated by reference in their entirety:

Provisional application Ser. No. 60/438,294, filed Jan. 7, 2003, entitled “Products free of volatile organic compounds (VOC's) promote health and eliminate chronic disease.”

Provisional application Ser. No. 60/438,779, filed Jan. 9, 2003, entitled “Products free of volatile organic compounds (VOC's) promote health, eliminate chronic diseases.”

Provisional Application No. 60/164,857, filed Nov. 12, 1999, entitled “Using Unusual Foods To Create Symptom-Free Diets For Persons With Food Allergies, Sensitivities And Intolerances” is hereby incorporated by reference.

International Application No. PCT/US00/31066, filed Nov. 13, 2000, entitled “Use Of Tropical Root Crops In Effective Intervention Strategies For Treating Difficult And Complex Cases And Chronic Diseases” is hereby incorporated in its entirety by reference.

application Ser. No. 09/889,133, PCT filed Nov. 13, 2000, now U.S. Pat. No. 6,632,461 B1, entitled “Use Of Tropical Root Crops In Effective Intervention Strategies For Treating Difficult And Complex Cases And Chronic Diseases” is hereby incorporated in its entirety by reference. application Ser. No. 10/682,546, filed Oct. 10, 2003, entitled “Use Of Tropical Root Crops In Effective Intervention Strategies For Treating Difficult And Complex Cases And Chronic Diseases” is hereby incorporated in its entirety by reference.

application Ser. No. 09/927,062, filed Aug. 10, 2001, entitled, Process of Using Sodium Silicate to Create Fire Retardant Products

application Ser. No. 08/818,195, filed Mar. 14, 1997, now U.S. Pat. No. 6,303,234

Provisional application Ser. No. 60/013,452 (filed Mar. 15, 1996, in the names of Karen M. Slimak and Robert A. Slimak, entitled “Using Sodium Silicate to Create Fire Retardant Products”)

Provisional application Ser. No. 60/040,709 (filed Mar. 14, 1997 in the names of Karen M. Slimak and Robert A. Slimak, entitled “Effectiveness of Sodium Silicate and Micro-layers of Silicon Oxide Glassy Films in Imparting Fire Retardant Properties and Moisture Resistance to Cellulosic Materials”)

Also incorporated in their entirety by reference are the references cited in U.S. Pat. No. 5,789,012, including the listed US patent documents, the listed foreign documents, and the other publications listed.

Slimak, K. M. 2003, The Effect Of Environmental Chemical Exposures On Autistic Children, Autism One Conference, May 24, 2003, Loyola University, Chicago, Ill.

Slimak, K. 2003. Reduction of autistic traits following dietary intervention and elimination of exposure to environmental substances. In Proceedings of 2003 International Symposium on Indoor Air Quality and Health Hazards, National Institute of Environmental Health Science, USA, and Architectural Institute of Japan, Jan. 8-11, 2003, Tokyo, Japan, vol 2, pp 206-216.

The following related patents are also hereby incorporated by reference in their entirety:

U.S. Pat. No. 5,234,706, issued Aug. 10, 1993, titled “Processes For Products From Potatoes, And Other Roots, Seeds And Fruit”,

U.S. Pat. No. 5,244,689, issued Sep. 14, 1993, titled “Flour, Bread, Milk, And Other Products From White Sweet Potatoes, Cassava, Edible Aroids, Amaranth, Yams, And Lotus”,

U.S. Pat. No. 5,204,137, issued Apr. 20, 1993, titled “Processes For Products From Sweet Potato”,

U.S. Pat. No. 4,911,943, issued Mar. 27, 1990, titled “Processes For Products From Amaranth”,

U.S. Pat. No. 4,946,703, issued Aug. 7, 1990, titled “Processes For Products From True Yams”,

U.S. Pat. No. 4,925,696, issued May 15, 1990, titled “Processes For Products From Malanga”,

U.S. Pat. No. 4,925,697, issued May 15, 1990, titled “Process For Products From Sweet Potato”,

U.S. Pat. No. 4,923,709, issued May 8, 1990, titled “Processes For Products From Cassava”,

U.S. Pat. No. 4,929,467, issued May 29, 1990, titled “Processes For Products From Lotus”,

PCT Patent W087/04599, filed Feb. 2, 1987, issued Aug. 1, 1988, titled “Flour, Bread, Milk, And Other Products From White Sweet Potatoes, Cassava, Edible Aroids, Amaranth, Yams, And Lotus”, and

Canadian Patent No. 1,313,602, filed on Feb. 2, 1987, issued February 16, 1993, titled “Flour, Bread, Milk, And Other Products From White Sweet Potatoes, Cassava, Edible Aroids, Amaranth, Yams, And Lotus”.

### BACKGROUND OF THE INVENTION

(1) Field of Invention

Long term exposure to volatile organic components (VOCs) has been linked to chronic diseases, conditions and symptoms such as diseases of the blood vessels, heart and/or lung; psychiatric disorders, diseases of the nervous system and genetic diseases, among others. Methods and products are disclosed to treat chronically ill patients as well as healthy patients to reduce or eliminate exposure to VOCs.

(2) Description of the Background

An important new method for studying a wide variety of diseases, conditions and symptoms has been described in U.S. Pat. No. 6,632,461 B1. The key component of this method involves the consistent, complete elimination of food-related symptoms in individuals suffering from a wide ranging array of diseases, disorders, conditions and symptoms followed by selective elimination of exposures to environmental substances.

It was because of this work, especially the last few years, that I learned how VOCs were traveling throughout the body causing superimposed reactions and causing the immune system to attack the body, how low the exposures needed to be stop the reactions, and that VOCs are not the cause of allergies, but are the cause of chronic diseases. Case studies from autistic children are really good subjects of study because they have so many symptoms and conditions, in addition to autism, that by studying autistic children, I was able to study essentially everything else, also the children are so uninhibited that it was very easy to tell exactly how they were being affected, and they are far more unbiased (uninfluenced by surroundings, conversations, etc.) than about any other group. They are essentially their own walking double blind study.

This methodology has been used to study approximately 1000 individuals grouped in a wide array of diseases, disorders, conditions and symptoms including but not limited to diseases, conditions and disorders listed in Table 1, and including but not limited to symptoms listed in Table 2.

TABLE 1
Chronic Diseases, Conditions, Disorders Studied

Blood, blood-forming organs, blood vessels:
Auto-immune haemolytic anaemia (antibodies form against the
person's own red blood cells)
Pernicious anemia (autoimmune disease, lowers b12 levels)
Diseases of the blood vessels and/or heart
Arteriosclerosis (inflammation and infection causing tissue
and plaque buildup in arteries)
Endocarditis (inflammation of the endocardium)
Hypertension (elevated blood pressure)
Migraine headaches (severe pain along nerves and blood vessels
spasms in head)
Polyarteritis nodosa (inflammatory attack on medium and small
arteries; any organ may be affected)
Thromboangiitis Obliterans (inflammation of blood vessels in
hands and feet, assoc w tobaco products).
Diseases of the lungs
Asthma (restricted airway flow)
Pulmonary Interstitial Fibrosis (inflammation causing lung
scarring (fibrosis) until oxygen unable to pass into lungs;
known environmental component)
Diseases of the digestive tract
Celiac sprue (auto-immune response to gluten; diarrhea,
bloating, gas, hair loss, muscles aches, cramps, skin lesions,
fatigue)
Crohn's Disease (inflammation of the bowel, often also asso-
ciated with infection) Crohn's Disease (inflammation of the
bowel, often also associated with infection)
Ulcerative colitis (chronic inflammatory intestinal disorder:
severe, persistent abdominal pain, sores of the bowel,
diarrhea, intestinal bleeding, weight loss, joint pain and
skin/eye problems)
Ulcers of mouth, esophagus and GI tract (caused by reactions
to food, chemicals, inflammatory processes)
Irritable bowel syndrome
Endocrine diseases
Diabetes (immune attack on cells in pancreas, or defects in
insulin action; progressive immune system attack occurs
throughout the body)
Hypoglycemia (low blood sugar)
Graves disease (immune system attacks thyroid gland, causes
overproduction of thyroid hormones
Hypothyroidism, Hashimoto's (immune system attacks thyroid
gland)
Sjogren's syndrome (immune system attack on fluid producing
glands causing loss of saliva and tears; progresses to other
immune system disorders)
Diseases of the eye
Uveitis (inflammation of middle tissues of the eye)
Bone, joint and muscle disease
Gout (arthritic joint inflammation plus uric acid crystals in
the joint) Gout (arthritic joint inflammation plus uric acid
crystals in the joint)
Paget's Disease (chronic bone inflammation: bone thickening,
softening, bowing, excessive bone destruction)
Rheumatoid arthritis (chronic destructive inflammatory attack
on joints; stiffness, swelling, pain)
Skin and connective tissue disorderss
Alopecia (autoimmune skin disease)
Ankylosing Spondylitis (inflammatory systemic disease,
affecting the spine and adjacent joints)v
Dermatomyositis (inflammatory muscle and skin disease)
Lupus (immune system attacks joint & muscles, skin (rash and
photosensitivity), membranes around heart or lungs, connective
tissue, blood (anemia, other abnormalities), kidneys & other
organs)
Myasthenia Gravis (autoimmune neuromuscular disease causing
weakness of skeletal muscles)
Pemphigus (auto-immune attack on skin and mucous membranes:
blistering large areas)
Periodontal disease (inflammation and infection of gums)
Psoriasis (chronic inflammatory attack on skin, T cells
(normally fight infection) attack skin and trigger other
immune responses)
Roseacea (irritation and inflammation of cheeks)
Scleroderma (immune system attack on connective tissue
disorder causing thickening and hardening of the skin, and
various parts of the body, including internal organs.)
Undifferentiated connective tissue disease (chronic inflam-
matory autoimmune diseases of connective tissue, generally
evolves into a specific disease such as lupus, rheumatoid
arthritis, etc)
Psychiatric disorders:
ADHD (attention deficit hyperactivity disorder)
Anorexia (refusal to eat)
Anxiety disorder
Bipolar Mood Disorder
Bulimia (binging and purging)
Depression
Obsessive-Compulsive Disorder
Panic Disorder
Unipolar Mood Disorder
Schizophrenia
Diseases of the nervous system
Alzheimer's Disease
Epilepsy (seizures)
Guillian Barre syndrome (inflammatory disease of the pe-
ripheral nerves; muscle weakness)
Multiple Sclerosis (inflammation of white matter of central
nervous system with demyelination; difficulty in walking,
numbness, loss of vision, tremor, incoordination, slurred
speech, paralysis, and a decline in ability to think, reason
and remember)
Parkinson's Disease (progressive disease of central nervous
system; loss of cells in substantia nigra)
ALS
Tourette's Syndrome (neurological condition characterized by
bodily jerking and contortions, vocalized grunts, obsessive-
compulsive behaviors, and strong food preferences and food
smelling)
Autism spectrum disorders (ASD), PDD, Asperger's syndrome,
other related conditions
Cancer
Infectious diseases
Peptic ulcer
Chronic Bronchitis
Lyme disease
Genetic diseases
Angelman's syndrome
Down's syndrome
Cystic Fibrosis
Marfan's syndrome
Porphyria
Addison's Disease
Alzheimer's Disease (possibly, several genes have been found)
Muscular Dystrophy (progressive weakness, degeneration of
voluntary muscles)

TABLE 2
Chronic Symptoms Studied Included the Following

abdominal pain, cramping
acid reflux, heartburn, spitting up
affection, lack of: unaffectionate
aggressive
anal irritation
anger, easily angered, angry outbursts
anxiety, anxious
appetite: poor, unable to eat enough
asthma, rapid breathing, difficulty breathing
attention deficit, inability to focus, zoning in and out
awareness of time: reduced
balance, poor, equilibrium: difficulties with
belching
biting
skin: tenderness, pain
bloating
body awareness: disinterest, low awareness
bowel function: fluctuating
breast tenderness
breath: off, odd, after taste, bad
skin: bumps, rashes, lesions
circulation: poor
climbing: seeking high places
clingy behavior
cognitive ability: reduced,
difficulty thinking clearly
cold hands, cold feet
comforted: not easily comforted
compliance, lack of
compulsive adherence to routine
congestion
constipation
coordination: poor, reduced
cough
dark circles; discoloration under eyes
depressed, pessimistic
destructive
diarrhea
drooling
ears, inner: discomfort, pain
eating habits, poor
eating disorders, bulimia
eczema
egocentric
emotionally unstable, fluctuating emotions
eye contact, lack of
social interaction: inappropriate,
overly social
social interaction: lacking, reduced, hesitant, uncomfortable
social interaction: non-responsive
social skills: delayed, lacking
staring at, focused on reflection
stools: discolored, mucous, undigested food
stubbornness, headstrong, wanting own way
stuttering, trouble with speech
sweaty, day or night
swelling, local area
facial muscles: difficulties using, especially mouth
fainting, loss of consciousness
fatigue
flushed
food cravings
eating disorders: food aversion
frustrated, easily overwhelmed
gagging
gas
head banging
headaches unrelated to
congestion
headaches: severe, migraine
headaches: sinus and congestion related
healing: slow ability to heal
heart beat: pounding, irregular, jumping, thumping
hiccups
hitting, pinching
hives
hunger: excessive, frequent
hurting self (pulling hair, hitting)
hyperactivity
hypertension
hypoglycemia
hyposensitivity: reduced perception of sensory stimuli
infections, frequent
infections: candidiasis, oral
thrush, yeast infection
intolerance of mistakes
irritability
infantile spasms
itching, scratching, stinging,
burning
joints: stiff, swollen
laughing, giggling, inappropriate
lethargic, underactive
loud screaming; profanity
memory: reduced
menstruation, difficulties
mouth: dry
muscle aches and pains
muscle spasms, cramping, tension
muscle tone, poor; muscle weakness
motor skills: lack of, delayed
fine motor, gross motor skills
nasal irritation, nose picking
nausea
nervous, tremulous, shaky
tantrums
temperature: high body
temperature; feeling hot; fever
temperature: low body
temperature, feeling cold
thirst: excessive
throat: constricted, sore
throwing objects
ulcers: mouth, esophagus, GI tract
upset, easily
urinary frequency, urgency, pain
verbal: reduced interest, curiosity
verbally non-responsive; non-reactive
obsessive, compulsive behaviors (OCD)
pain: joint and bone
pain: other
panic attacks
peripheral vision, exclusive use
pica: eating dirt, sand, non-food
play: reduced interest, ability; hesitance
post nasal drip, runny nose
posture: rigidity, body stiffening resistance to change
rituals: clothing related
rituals: food-related
rituals: insisting on, prolonging car rides
rituals: play
rituals: repetitive physical motions
rituals: repetitive play
rituals: verbal
rituals: writing
rubbing, face, skin, repeated
seizures
self help skills: reduced interest
self-stimulation: auditory
self-stimulation: physical
self-stimulation: verbal, chatter
self-stimulation: visual
sensitivity: light
sensitivity: noise
sensitivity: tactile
sensitivity: to pain
sensitivity: to pressure (atmospheric)
sensitivity: to smells
skin color (red, pale)
skin: dry
skin: blisters
sleep: active, restless during sleep
sleep: crying, screaming
sleep: easily roused, startled
sleep: insomnia, inability to fall asleep, sleeplessness
sleep: nightmares, bad dreams
sleep: poor quality, difficulties sleeping
smelling: repeated, inappropriate, indiscriminant
social interaction: avoidance, upset in public places
social interaction: inability to relate to others
violent outbursts
vision: difficulties associated with vision
vomiting
vulvar irritation, itching, pain
walking: difficulty, unable to walk
weight: difficulty with, underweight, overweight
wetting bed or clothing, toilet training
whiny behavior
withdrawn
writing: reduced interest, aversion

The basic method of study was the same for each group and all individuals. Food-related symptoms were eliminated and optimal nutrition was achieved with a 7-day rotation diet of tropical root crops as the primary carbohydrate and then combined with unusual meats, fats and vegetables to provide a nutritionally well-balanced non-reactive diet, also called ‘the Special Foods Diet’. In the absence of otherwise confounding and confusing food-related symptoms, changes in the remaining symptoms were then observed as chemical exposures were selectively removed. Various environmental exposures were eliminated and the corresponding changes in remaining symptoms were observed. As the types of exposures fluctuated and the levels of exposure fluctuated, observation of fluctuations in symptoms continued over periods of time, ranging from 4 weeks to 3 years per person.

It is important to note that environmental exposures were reduced to below ambient levels and then allowed to return to ambient exposure levels. The environmental exposure levels were never increased. The effects of reducing exposures below average or typical exposure levels in everyday environments was observed in individuals who were experiencing chronic symptoms, conditions, diseases, disorders.

Regardless of diagnosis, each subject described his/her complete set of symptoms and complaints that was being experienced, including those that were not related to a diagnosis. Each symptom/complaint was rated on a scale of 1-10, with 10 being the most extreme expression of that symptom. All symptoms/complaints each subject was experiencing were studied in order to discover more fully the extent of the effects of environmental exposures on each person studied.

The individuals studied suffered from chronic symptoms, conditions, diseases, disorders and experienced unrelieved and generally uncomfortable symptoms that left them unsatisfied with the results of conventional treatment and urgently seeking relief from their discomfort. The conditions were generally in the moderate to severe range, but not at a stage where significant cell death or irreparable tissue damage had occurred. Most subjects were cared for by family members in their homes.

Exposure to Volatile Organic Compounds—the Underlying Cause of Chronic Disease:

Important results from long-term study of these many groups of individuals suffering from chronic conditions, diseases, disorders and symptoms include the following (this describes the results achieved after full elimination of all fold-related symptoms; these were only possible to observe under conditions of complete food-related symptom elimination):
1 VOC exposure sole cause of symptoms: Exposure to volatile organic compounds (VOCs) was found to be essentially the sole cause of the symptoms experienced by each individual studied. Volatile organic compounds were responsible for virtually all of the symptoms remaining after the elimination of food-related symptoms through the above-described diet.
2 More than chronic conditions/diseases/disorders eliminated: In each person studied, the symptoms associated with chronic conditions/diseases/ disorders as well as essentially all additional symptoms not associated with that condition/disease/disorder were caused by exposure to volatile organic compounds. Consistently, the effects of VOC exposure extended well beyond the symptoms associated with a particular diagnosis, and included essentially all of the symptoms the person had been experiencing. It would have been very misleading to observe only diagnosis-related symptoms.

3 Symptoms/conditions/diseases/disorders disappeared, reappeared as VOC- exposures were eliminated, returned: Specific symptoms as well as complete conditions/diseases/disorders were routinely and fully eliminated by eliminating exposure to VOCs. Symptom/condition/disease/disorder elimination was sustained when the eliminated VOC exposure was maintained, and finally symptoms were found to recur fully following re-exposure to volatile organic compounds.

4 Direct correlation between VOC exposures and symptoms/conditions/diseases/disorders: A one-to-one relationship between symptoms/conditions/disease/disorder and exposure to volatile organic compounds was found. A very high correlation between exposure to volatile organic compounds and symptoms was observed (p<0.000) in one study of 49 subjects. Symptoms and chronic conditions were present when there was exposure to volatile organic compounds, and the symptoms and chronic conditions were fully absent when there was no exposure to volatile organic compounds. This is essentially a one-to-one correlation between VOC exposure and the above listed symptoms and conditions studied.

5 Mild symptoms: In individuals experiencing mild symptoms and in individuals who were experiencing the early stages of a particular condition (generally prior to diagnosis), partial removal of exposures to volatile organic compounds was sufficient to achieve and sustain elimination of the symptoms and conditions.
6 Complete elimination of VOC exposure necessary: However, for individuals experiencing moderate to severe symptoms and progressing to the point of diagnosis and beyond, virtually complete, total elimination of exposure to volatile organic compounds was required to achieve sustained symptom relief and to recover. For individuals experiencing moderate to severe symptoms/conditions/diseases/disorders, symptoms/conditions/diseases/disorders returned whenever there was re-exposure to even extremely low levels of volatile organic compounds. Many systems in the bodies of the subjects were affected by low levels of even a few types of volatile organic compounds. Strong reactions occurred at extremely low levels, these levels were at and below detection limits for ability to smell, the part per trillion (ppt), part per quadrillion (ppq) levels and below for VOCs as a group.
7 VOC effects followed distinct time-related patterns: Following a VOC exposure, the appearance of symptoms followed distinct time-related patterns. There was a time to onset of symptoms, a period of heightened symptoms, and a period of gradually declining symptoms to the point of complete disappearance. A single exposure was responsible for reactions lasting from 4 days to 2-3 months; the reaction duration, from point of exposure to return to complete symptom elimination, for most individuals was 4 days to 2 weeks.
8 For each individual VOC effects consistent and repeatable: For each person studied, a specific exposure triggered a unique array of symptoms and a severity level for each symptom in the array. This pattern was consistent and repeatable for each subject whenever the same exposure occurred. This unique pattern of symptoms and severities, representing a unique response of that person's immune system to that particular exposure, was as unique and consistent as a fingerprint
Because of the time of symptom duration and the large number of VOC exposures, prior to the study participating individuals had experienced overlapping reactions that caused symptom levels that fluctuated as exposures changed, but essentially never ended in a manner that clearly indicated the VOC cause and reaction patterns of the effects.
9 VOC effects unique for each individual: The same exposure triggered a different array of symptoms and severities in each person studied. Among the individuals studied, although each individual responded consistently and repeatedly in their own unique way, there was no consistently similar response to a specific exposure among the individuals studied; ie there was no correlation between a VOC exposure and any one symptom or set of symptoms.
10 Improvements in enzyme levels, chronic infections, temperament, immune function: Prolonged elimination of VOC exposures caused unanticipated but important changes to the groups of individuals studied. Although the only treatments applied were the above described diet and elimination of VOC exposure, surprisingly in the individuals studied chronic infections were spontaneously resolved, enzyme levels spontaneously returned to normal, there was a temporary spike in levels of metals in blood, urine and stool samples (for example, levels of mercury, aluminum, copper) that appeared to occur as enzyme levels returned to normal, improvements in temperament and disposition were profound, and there was a shift in immune function away from a chronic, continually hyper-reactive state.
11 Addiction: Consistently, strong addiction to volatile organic compounds was observed. For many, it was discovered that seeking out and maintaining exposure to volatile organic compounds was actually the focus of their lives. This became obvious after eliminating food-related symptoms as described above and subsequently sequentially eliminating VOC exposures. Seeking new VOC exposures was particularly strong, very obvious, and often frantic at the end of a reaction cycle. Withdrawal was similar to other types of withdrawal such as withdrawal from alcohol, and generally lasted the length of one reaction cycle. When the diet was followed carefully and when there was simultaneous consistent elimination of exposure to VOCs, the cravings were eliminated. The cravings were generally eliminated within two weeks to two months. There was no continued craving unless and until there was a new exposure to VOCs.
12 Recovery: It was possible to achieve and study long term VOC avoidance. When consistent elimination of VOC exposure was maintained long enough, the immune system slowly became less and less reactive. Periodic VOC exposures and their triggering of the immune system mediated responses (ie, reactions), as frequently as monthly or bi-weekly, were sufficient stop the slow process of immune system recovery. Depending on severity of the initial symptoms/conditions/diseases/disorders, between 6 months (in mildest cases studied) to 5-10 years (in severest cases studied) were required to achieve complete recovery. Recovery is defined as being fully symptom-free, the original condition/disease/disorder not present, able to return to a normal diet without restrictions, and able to experience occasional VOC exposures without experiencing associated effects, addictive behaviors or cravings. Recovery occurred consistently in individuals who followed the above-described diet and achieved complete, sustained elimination of VOC exposures.
13 Serious injuries: Individuals experiencing birth-related injuries, and other serious injuries that typically cause long term, slowly developing symptoms (in addition to the acute injuries) were studied using the above identified protocol. It was found that in addition to the injuries, there had been a strong immune system response to that injury that continued long after the acute event; many times the immune system response caused greater damage than the initial injury. The progressive, continuing immune system response was halted when the above described protocol was applied. These individuals experienced the changes in immune function, enzyme levels, temperament, metals, elimination of chronic infections as other individuals in the study. The immune mediated deterioration was halted, and a gradual recovery was experienced that was limited only by the ability of the injured tissue to recover. As long as the damage caused by immune system related effects had not progressed to the point that the damage was irreversible, slow reversal of these effects was observed.
14 Genetic diseases/disorders/conditions: individuals experiencing genetic diseases/disorders/conditions were subjected to the above described protocol. It was found that surprisingly, essentially all of the non-structural effects and behaviors were eliminated. Only structural physical characteristics were unaffected. This indicates that there is an immune response to genetic-based conditions, and this immune response has not been separated from the genetic condition/disease/disorder. The immune component was halted and eliminated in the individuals studied. Some, such as pemphigus vulgaris were eliminated completely, while others, such as Angelman's syndrome, all except structural physical characteristics were eliminated.
15 Immune response: The response to VOC exposures did not follow the pattern of a toxic effect, in which severity was dose dependent. The subjects' responses to VOC exposure followed the pattern of an immune system response, which is very strong response at very low levels. The VOC concentration above that low level is essentially unimportant since any exposure above the trigger concentration triggers a full response.
16 Study of non-ill individuals: VOC exposure was also eliminated in individuals who were essentially without symptoms. In these individuals sense of smell became acute, and subjects were able to detect VOC exposures at ppt and ppq levels, and spontaneously avoided further exposures. These subjects reported exposure-related symptoms such as headaches, stomach aches, mood changes and the like.

Conclusions from the observations and data gathered:
1 VOC exposure is responsible for essentially all of the chronic symptoms, conditions, diseases and disorders experienced by individuals.
2 The symptoms associated with chronic conditions/diseases/disorders are, in fact, strong, superimposed, ongoing, individually unique reactions of the immune system to thousands of simultaneous and continuing VOC exposures.

3 Chronic conditions/diseases/disorders are merely collections of symptoms. Symptoms are reactions (immune system responses), often superimposed, that are continued and sustained by repeated exposure to thousands of volatile organic compounds.

4 Immune system responses to serious injuries and genetic defects/conditions/diseases/disorders are halted and eliminated by simultaneously following the Special Foods Diet and by complete elimination of VOC exposures.

5 Effects of VOC exposure on the nervous system was particularly strong. Virtually every aspect of neurological function was found to be effected strongly by VOC exposure, including but not limited to seizures, cognitive functions, control of muscles and organs, ability to speak, sensory sensitivity, OCD, temperament, ability to function in social contexts, addictive behaviors, effects on impulse control and the like.

6 To eliminate moderate to serious conditions/diseases/disorders VOC exposure must be completely eliminated. VOC exposure levels at the part per quadrillion level are required. Since VOC exposures frequently contain 1000 to 10000 different chemicals, individual chemical levels need to be below the 0.0001 part per quadrillion level. This is below the ability of analytical chemistry to measure, and thus below the ability to know when the VOC levels are low enough. The best, preferred and essentially only way is to make sure that no VOC contamination is possible.

7 There appears to be no need to search for inherent defects associated with the observed effects of volatile organic compounds on humans. Following elimination of VOC exposure, symptoms disappeared, infections were eliminated, enzyme levels returned to normal, and immune system function returned to normal. No apparent symptom, difficulty or other problem or complaint remained following sustained elimination of VOC exposure.

8 A myriad number of symptoms/conditions/diseases/disorders all are caused by the same set of substances, VOCs. This does not point to an inherent defect.

9 From careful study of individuals by the above methods, and noting the ability of the immune system and enzyme levels to return to normal and the fact that volatile organic compounds have long been present in the environment, it is apparent that the immune system can handle without symptoms, even high levels of volatile organic compounds without symptoms or adverse effects, provided that the exposures are not chronic. In recovered individuals, occasional exposure to even high VOC levels was handled easily without return to the previous symptoms/conditions/diseases/disorders, as long as exposure to high VOC levels occured only occasionally, and the rest of the time, the VOC levels were extremely low. The problems of millions has resulted from chronic exposure to high VOC levels.

10 It is sad realize that there appears to be nothing wrong with virtually all of the millions of individuals who are ill and dying from chronic conditions/diseases/disorders. The unnecessary cost amounts to billions upon billions of dollars in lost wages and medical costs. Millions are needlessly suffering. Elimination of VOC exposure will save millions of lives, untold billions of dollars and virtually eliminate chronic disease.

11 Humans not intended for chronic VOC exposure at high levels. The result is chronic disease, conditions, disorders

Effects of VOC exposure are not limited to specific chronic conditions/diseases/disorders but extend to virtually all symptoms experienced by an individual.

Thus work of other investigators who have been conducting research on specific conditions/diseases/disorders has not been helpful in identifying the underlying cause. Rather such research has actually been directing away from the underlying cause, and instead partially describing the process of deterioration that occurs as the immune system reactions become increasingly strong at increasingly lower levels, as enzymes become increasingly impaired, and as chronic infections become increasingly severe.

Until the instant invention, treatment has focused on symptom alleviation while leaving the basic process of deterioration (immune system attacks, increasingly impaired enzyme function, decreased ability to fight and eliminate infection) unaddressed.

These discoveries lead to finally understanding of the underlying cause of chronic disease. This is important because, as is described below, chronic diseases are increasing rapidly, and have increased especially in the past 10 years. At present, approximately 50% of the US population suffer from one or more chronic diseases.

Natural populations that are well-fed, sheltered, and are provided all the basic necessities of life, are sleek, healthy, and essentially disease free. The mark of stressed populations is susceptibility to disease.

For individuals living in developed countries, people are well-fed, sheltered, and are provided all the basic necessities of life, they should be sleek, healthy, and essentially disease free, instead there is a rapid increases in chronic diseases, and the high levels of chronic disease that now occur is alarming, and cause for great concern.

Volatile Organic Compounds:
Volatile and moderately volatile organic compounds (all referred to throughout as volatile organic compounds, VOC's) are a large group of organic molecules, generally containing 100 carbon atoms or less, also containing hydrogen, and possibly oxygen, chlorine, nitrogen, and various other functional groups; although some consist solely of carbon and hydrogen, VOC's may be aromatic or aliphatic, saturated or unsaturated, or combinations.
Included in volatile organic compounds are sugar molecules, fat molecules, amino acids, building blocks of DNA, proteins, and the like.
The instant application deals with the large group of volatile organic compounds that remain, ie, do not have a defined role in the body. Applicant has discovered the terrible effects of VOC's that enter the body, but have no role once there, hence non-essential.
There are 70,000 to 100,000 non-essential volatile organic compounds known. In the average home at any given moment, there are approximately 1,000 different volatile organic molecules inhaled with each breath, and the composition and concentrations change almost constantly. The composition and concentration of volatile organic compounds that are breathed in vary as a person walks through rooms, eats, sits in front of the computer, showers, shaves, and the like. The average 1 ppm level in indoor environments means that approximately 14 million billion of these constantly changing molecules enter the body with every breath.
There is in that large number, the proper size and shape to mimic ever essential compound, to interfere with every enzyme function, to occupy and inactivate sites intended to find and remove infectious organisms, and to trigger autoimmune system attacks throughout the body. This constant shift in molecules at these levels is one of the factors that causes so much trouble.
These substances are similar enough to their ‘essential’ counterparts that they are taken up by active and passive means, surge into the blood stream, and enter every cell in the body.

How VOC Exposure Causes Chronic Disease:

In order to know what to do about this serious problem, it is necessary to understand how it is possible for VOC exposure to result in chronic symptoms/conditions/diseases/disorders, in otherwise healthy individuals. As part of the instant invention, applicant's description of the underlying process is summarized below:

1) The immune system is unable to distinguish between the above non-essential volatile organic compounds and invading bacteria, viruses, and other organisms. The exterior markers by which the immune system recognizes organisms, if torn away from the organism, are volatile organic compounds.
There are approximately 1500 CFU (colony forming units, i.e., viable organisms) or less per cubic meter in the air. This is approximately 1 viable organism per breath; the organisms begin to multiply, are encountered and removed at very low levels. The immune system can be triggered fully at these incredibly low levels.
So, if the immune system responds to volatile organic compounds as if they are invading hoards of bacteria, molds, viruses, etc, as it appears is true, it is not hard to realize that continual exposure to volatile organic compounds at billions per liter instead of 1-100 per liter, could overwhelm the immune system.

2) It appears that volatile organic compounds, the immune system, and ability to fight infection are intertwined and virtually inseparable.
In the natural environment, infectious agents e.g. mold spores, are rarely encountered without accompanying odors (e.g., musty odors for mold) also being present. This appears not to be accidental and is a vital part of predator/prey and interspecies competition, especially on the micro level, as part of microbial ‘warfare’.
One to two viable organisms accompanied by billions of ‘fake’ organisms, e.g., volatile organic compounds, is an important strategy at the micro level. The immune system of a large organism (the large organism is the prey from the perspective of a microbial invader), encountering all this, will be far less able to find and remove the few viable organisms that are unrecognizable as different, hiding among all the VOC's. (This would be like a queen bee hidden among a swarm of bees; how could one find the queen if one could not tell the queen from the workers?) In addition, the resources of the immune system, i.e, the ability to detect and remove foreign agents, although impressive, is inherently limited, i.e., the number of active sites available although large, is not infinite. The VOC's occupy the active sites intended for infections organisms, thus reducing the active sites available for fighting infection and other foreign agents; this reduces infection-fighting capability many times over. The strategy of the ‘invaders’, then, would be to 1) use the VOC's as a smoke screen and to direct the immune system attack away from the invading organism. 2) Induce the immune system to waste active sites and immune system resources to the point of virtually running out. This on a military scale was a tactic used in World War II. In England, for example, fields of cardboard planes were built to draw away the enemy fire from the real planes. I am suggesting this as well as virtually running out of ammunition. Now this inventor has observed evidence of this occurring at the microbial level.

3) VOC's are addictive; also with increasingly higher levels of VOC's in the ambient environment, the less one is able to detect the scents because the olfactory pits in the nose swell close and the myriad number of strong smells confuse the nervous system and they are no longer detected and distinguished. This makes it possible to tolerate increasingly higher VOC levels without becoming aware of adverse effects. This would be the third strategy in microbial warfare. As VOC exposure increases, the ability to detect (smell) those that are preferred (pleasant scents) diminishes, gradually requiring higher levels of exposure as the sense of smell gradually diminishes. Generally, gradually and unknowingly, individuals come to rely on an initial sense of pleasure, euphoria that frequently occurs with early stages of reactions following VOC exposure. Add to this the incredible intelligence of humans, and the time and money we now have to pursue things that give us pleasure. It is very likely that the intelligence of humans is itself essentially taken over. Instead of using our intelligence to protect ourselves, our intelligence is used to find ways to increase our levels of exposure to volatile organic compounds.
The inventor has seen this clearly with the 49 autistic children who have been carefully studied. Strongly addicted behaviors have been observed by this investigator were observed consistently in autistic children autistic children consistently exhibited behaviors of strongly addicted individuals. In a clean room free of VOCs, the autistic child spent every moment in the first few weeks, frantically trying to find, increase or create exposure to VOC's, to the exclusion of everything else. It was only possible to observe this behavior in a VOC clean room; in typical home and school environments the behaviors were more subtle and easily misinterpreted as ‘simply being autistic’.
This investigator has observed that the same process occurs in the general population.

4) As a result of this (1-3, above), in the presence of constant exposure to VOC's, the capability of the immune system to fight infection is seriously compromised. This occurs simply because of the sheer numbers of VOC molecules present in today's ambient indoor environment; the high numbers of VOCs occupy the active sites intended for invading microbes, leaving few available to fight infection. Even though antibiotics are available, chronic infections become established. These result in patches of irritated, inflamed tissue on the macro and micro level throughout the body; the array (specific locations of the patches of irritation and inflammation) is unique to each individual. The infections never completely clear, in spite of the use of antibiotics. This should be a cause for concern, because it indicates that the immune system is unable to clear even the small pockets of infection. In these irritated areas functional groups are more exposed and more easily accessible.

5) Eventually the immune system begins to be able to recognize specific VOC's, and develops second level responses involving antibody production, and so forth. The result is that at this time, the immune system produces a variety of substances, specifically configured for a particular volatile organic compound that travel throughout the body searching for that compound. It is at this point that immune system-related symptoms begin to occur.
Unfortunately, the VOC's that triggered the immune system response are not spread around the body as are patches of infected tissue. VOC's travel in a bolus in the blood stream. They trigger the immune system response at the point where they enter the body, generally through the lungs. The immune system response, briefly described in 5) above, travels throughout the body in the bloodstream along with the VOC's not removed.
There are no patches of VOC's to find. What does the immune system find? The answer is the result of molecular mimicry.

6) Agents of the immune system, designed specifically for particular VOC's, travel throughout the body, find and attack functional groups that are similar in size and shape to those VOC's.
This is caused by molecular mimicry. In spite of assertions otherwise, the immune system is really not very specific. Any particular response, designed for one particular chemical, can also attack anywhere between 12 and 50 other compounds. In fact, any molecule, or part of a molecule that is similar in size, shape and polarity, will fit into the active site, regardless of the actual molecular composition.
This is so important. VOC's trigger immune system antibody responses, but are not found and removed. Instead the immune system antibody response attacks exposed functional groups throughout the body that are similar chemically in some way to the original VOC. In effect, the triggered immune response, seeking to respond to the presence of VOCs in the body also attach to functional groups in healthy cells due to the inability of the immune system to differentiate between a healthy functional site in the body and free-ranging VOCs, and immune system attacks any similar functional sites that are chemically, structurally similar to the target VOC, because of molecular mimicry. The result is a steady immune system attack on the body because of constant VOC exposures leading to the creation of chronic diseases, conditions and symptoms as exemplified in Table 1, and a simultaneous reduction in the ability of the immune system to fight infection,.
The immune system continues to develop responses to compound after compound, and the responses become stronger and stronger.
Because each person develops immune system responses to chemicals in different order. The immune system attacks different parts of the body, and different organ systems, in various individuals.
In addition, the immune system attacks micro-patches of irritation and inflammation caused by the chronic infections that cannot be completely eliminated. This is another way that causes symptoms that are unique to each individual - the patches of irritation are unique to each person.
This VOC triggered immune system attack continues essentially without end, and progresses as a disease, condition or symptom progresses and continues. This is because new immune system responses are triggered with every breath the person takes.
This is also one reason that in any illness, even a serious one, there are better days and worse days. The better days invariably follow days where exposures are reduced for some reason, so there are fewer immune system reactions.

7) VOC's that are not removed immediately by the immune system travel in the blood stream and pass through cell membranes by active and passive means, entering every cell in the body by the billions. Inside the cells they ‘bounce around’ rapidly. It takes a very short amount of time to crash into primary or secondary active sites, effectively disabling that enzyme. This happens ‘by the billions’.
The result is continual lowering of enzyme levels over time, until genetic variation begins to show.
Although it may appear that genes are dysfunctional, instead VOC-related enzyme disablement is outpacing the natural process of anabolism and catabolism.
Enzyme disablement appears to occur at random. Because of the large numbers of different VOC's involved, the capability exists to gradually disable most enzymes at essentially the same time. Thus in addition to differences in particular chemical exposure patterns, genetic deficits begin to appear, according to the particular genetic makeup of each individual.
This explains the apparent epidemic-like increase in genetic disorders. When one thinks about it, there cannot be a genetic epidemic.

8) Lists of the following chemicals are essentially the same; all are volatile organic compounds. Once a chemical is immuno-activated, the immune system responds identically, regardless of source.
Haptens (external markers on the exterior of microbial organisms, the way the immune system recognizes invading organisms)
Pheromones and chyromones (chemicals for defense, communication and competition),
Moldy musty smells (molds),
Natural and synthetic flavor and aroma compounds, including those in food
Volatile organic compounds of indoor air pollution,
Outgassing chemicals from most petroleum based products

10) Volatile organic compounds are responsible for reducing the ability to fight infection, triggering continual attacks of the autoimmune system thereby causing and continuing most of the chronic diseases mankind suffers from, and also disabling the enzymes. This causes progressive weakening of the individual and ultimately loss of life.
Because the exposures are continual in the environment of today, the ability to detect by smell and avoid is seriously diminished. The self protective mechanism has been lost.
The progression tracks a unique path in each person, making the patterns difficult to discern.
The continual exposure to volatile organic compounds, makes it virtually impossible to perform cause and effect assessments. Most individuals who have proceeded to the stage of chronic disease are experiencing thousands of simultaneous reactions that are superimposed, each lasting between 4 days and 2 weeks.

11) Everything appears to be reversible.
Unless the disease or condition has progressed to the point of complete cell death, and irreversible damage, the symptoms, condition and basis for the diagnoses quickly disappear when exposure ends.
In the individuals this investigator has studied, there has not appeared to be anything inherently wrong with the subjects.
Instead of finding a defect of some kind in each person, I found that the mechanism was actually an unavoidable, inevitable, logical result of chronic exposure to levels of volatile organic compounds that are far higher than the body was designed for.

12) The supporting data, are precisely consistent with 1-11 above. That is, complete, sudden drops in symptoms when exposure to VOCs is eliminated. This generally occurs at the end of a typical reaction period for each person, 4 days to 2 weeks. Among a group with a particular condition, the numbers are essentially 100 percent relief from symptoms in 100 percent of the individuals studied.
This is because the immune system cannot be triggered when there is no exposure. Ones immune system cannot react to something one is not around.
I have found the human body responds consistently to VOC exposure elimination with abrupt disappearance of the symptoms/conditions/diseases/disorders, the person was experiencing.
This is very different from that experienced by investigators who seek to treat the symptoms of chronic disease. Until now, investigators have been limited to studying treatments for individuals who are continuing to be exposed and to experience simultaneous superimposed reactions. Because the underlying triggers remained, variation in responses was inevitable.
This variation in responses has been eliminated with the treatment of the instant invention.

Treatment for Chronic Disease: Eliminate Voc Exposure

How often, does one eliminate chronic disease on a large scale? How does one go about the process of preventing chronic disease currently and in the future? The purpose of the instant invention is to describe means for eliminating chronic disease on a large scale, treating severely ill individuals and preventing chronic disease in the future.

Development of effective treatment strategies began by considering the following:
1 Individuals suffering from chronic conditions/diseases/disorders required essentially complete, sustained elimination of VOC exposure in order to eliminate their symptoms, their disease, and eventually achieve full recovery. The target levels for VOCs were: at or below 1 part per quadrillion for groups of VOCs, and at or below 0.001 part per quadrillion for single VOCs. They can most effectively be treated in controlled environments, such as by removing or shielding the VOCs emmissions from the patient. In one embodiment of the invention VOC emitting painted walls are coated with a material comprising an impermeable barrier, such as a glass or a glass precursor. Such a treatement can be effective in a room or section of a home in which a patient is confined. However, in other embodiments, special care in selecting materials of construction which do not emit VOCs can be used. If VOC presence is required, e.g., by the use of polymeric tubing in syringes, catheters or intravenous tubes or appliances, the surfaces of the tubing exposed to the patient, e.g., the interior of an intravenous tube, can be shielded with a material effective to block VOCs. In such a manner sanitariums, hospitals, health centers, nursing homes and other such buildings can be constructed with a design so as not to emit VOCs within their interiors.

2 To avoid chronic conditions/diseases/disorders, sources of chronic exposure to VOCs must be eliminated. Such can be achieved as above, e.g. eliminating the sources of VOCs emmissions, or coating/shielding the source of the emission.

3 Natural and/or organic materials of biological origin contain VOC levels that are too high. This means one cannot achieve part per quadrillion levels of VOCs by switching from synthetic to natural materials of biological origin. Thus not adding VOCs does not mean VOCs are not present. Higher levels exist in essentially all biological materials and essentially all organic materials, regardless of natural or synthetic origin. It is thus within the scope of the invention to substitute inorganic materials, metal, ceramics, glasses and composites for the almost everpresent polymers (plastics) from which VOCs emissions occur, as well as to coat/shield such materials to prevent emission.

4 Analytical measurements do not reach to the part per quadrillion levels. Thus analytical measurements showing, ND, ie, none detected, would contain VOCs at levels high enough to trigger a full immune response.

5 Chronic exposure to VOCs that have no role in the body cause chronic symptoms/conditions/diseases/disorders regardless of origin; thus natural and synthetic VOCs are indistinguishable in terms of how they affect the body.

6 VOC levels indoors and outside are magnitudes higher than they were decades ago. In the past, large amounts of time were spent outdoors engaging in vigorous activity in relatively pristine outdoor environments and bedrooms were generally unheated. This limited VOC exposures from wood stoves, etc and the activity levels outdoors provided opportunity for the VOC levels in the blood to be reduced.

7 Currently the patterns have switched, most time is spent indoors (with the highest VOC exposure levels) and the time spent outdoors is relatively brief. In most places the VOC levels outdoors are now too high to allow blood VOC levels to drop as they used to and even routine activities, such as shopping provide new souces of VOC exposure from shops, malls and similar commercial environments.

In order to avoid the misery, shortened life span, and staggering economic penalties associated with symptoms/conditions/diseases/disorders, the body needs to spend the bulk of its time in a pristine environment that is essentially free from all exposures to VOCs. This can be interspersed with occasional exposures to high levels, but only occasionally. Continuous exposures to VOCs leads most certainly to the health problems that have been described in the present application.

Although most definitely outdoor air pollution levels must be reduced, this is relatively little help since most people now spend 90% or more of their time indoors.

Thus the pristine environment must be created indoors.

Virtually all of the materials and products introduced to the indoor environment in the past 50-60 years have contributed to the current high levels of chronic disease. This includes but is not limited to furniture and furnishings, toys, computers, food handling and packaging, paints, plastics, clothing, bedding, home construction techniques, building construction techniques, heating and air conditioning systems, techniques for administering medications and treatments, treatment and delivery of water.

For example, volatile organic compounds are present in plastics as unattached molecules in the resin; they (VOCs) are raw materials, reactants, solvents, catalysts, additives such as plasticizers, intermediate reaction products and the like. All are present in the final product since, chemically, no reaction can ever go to 100% completion. These substances are generally present at part per million levels and higher, especially when combined body burden from all sources is considered. Although there is an initial surge of outgassing in a new product, there is also a long term, steady release of these VOCs at levels far above the part per quadrillion level.

The continued exposure to combined VOC levels overwhelm the immune system, slowly poisoning the body, causing chronic diseases, and ultimately shutting down organ function and shutting down enzymes. The current VOC exposures are high enough to overwhelm and cause moderate to severe chronic disease in approximately half of the US population alone.

The reason for this is the fact that VOC exposure is consistent and relentless in all possible areas of exposure. Levels of contamination that are beyond the body's ability to cope and recover from are now found in air, water, food, clothing, medication, homes, businesses, products, goods, and virtually every material present in our lives.

For example:
VOCs are added to foods and then introduced into the body (from plastic wrap, plastic oven ware, plastic food packaging, food processing techniques, food milling techniques, dishes, cooking utensils),
VOCs levels in foods themselves have increased, and foods are now much more highly flavored than in past decades (flavor and aroma compounds in spices, sauces, processing techniques to intensify flavor, flavoring packets and the like),
VOCs are added to medications, pharmaceuticals and supplements and then introduced into the body (capsules, dyes, coatings, most inert ingredients, plastic containers, tubing, packaging, VOCs from plastics and other sources incorporated during manufacturing),
VOCs are released directly to the blood stream (from IV tubing and the like, preservatives and other ingredients in IVs),
VOCs are contacted to the skin and taken into the body cutaneously (perfumes, scented lotions, cosmetics and similar products, nail polishes and removers, soaps, detergents, ointments and creams, volatile organic compounds from clothing (strand treating, dying, finishing, synthetic and natural fibers), substances in medications applied to the skin, and the like),
VOCs are added to and present in water and then introduced to the body by drinking and skin contact (water chlorination, plastic water pipes and plastic water delivery systems, plastic containers, plastic dishes and utensils),
VOCs are released to the air and inhaled directly into the lungs (showers, release of VOCs from furniture, furnishings, bedding, computers, games, telephones and the like, interior surfaces of buildings and homes, carpets, toys, paints, wallpaper, wall treatments, floor treatments, including finishes and sealants applied to stone and other natural materials, polishes, waxes, sealants, polishes, room fresheners, electrical cords, inhaled gases from medical procedures including breathing treatments, during administration of anesthesia, air and oxygen with and without humidification),
VOCs are released to the a, find their way into rooms and hallways, cabinet interiors and closets from interior spaces of buildings (between walls, in attics, materials of inner and outer walls, ceiling, roof construction, materials passing through these areas) and ultimately are inhaled into the lungs (insulations, foam sealants, caulks, adhesives, sealants, fire retardants, pesticides, electrical cords and conduit and similar materials, treated woods, pressed wood, strand board, particle board, masonite, vapor barriers, styrofoams, rust treatments, lubricants, concrete additives, machine oil coatings of metals, and the like)
VOCs are released to the circulating air and inhaled into the lungs by heating and air conditioning systems (VOCs from mold, VOCs from insulating materials, from oil coatings on metals, recirculated from contaminated areas of buildings, from filtering materials including resins, materials of ductwork construction, from plastics in adhesives, caulking, sealants,
Mold also produces volatile organic compounds as do foods and essentially all biological materials. The cellular structure of wood provides openings in which mold can grow, this causes dual issue of VOCs from both wood itself and from mold.

Add to this the fact that air pollution levels in the outdoor environments are high enough to create visible haze that can at times cover most of the US, most jogging trails parallel streets (where automobile exhaust is highest), and warnings to stay indoors to avoid the air pollution are common, means there is now no environment that is unpolluted enough.

It is the combined exposure level that needs to be reduced to the part per quadrillion level and below.

In previously studies, this investigator had observed that when diet and environmental exposures were reduced, some individuals experienced drops in symptoms. It was very difficult to achieve a zero symptom level and essentially impossible to maintain a zero symptom level, to stop the continual triggering of the immune system, and to achieve full recoveries. Almost anything could trigger new symptoms.

It was only the recent opportunity to observe and study over long periods of time (weeks and months) the reactions of moderately to severely chronically ill individuals in clean rooms in which the VOC levels in the clean rooms were progressively lowered and then maintained at essentially ‘0’ exposure in all areas, that made it possible for this investigator to understand and clearly see the levels to which VOCs exposure needed to be reduced and why previously it had been only possible to achieve fluctuating symptom levels. It was here in VOC-free clean rooms that this investigator was finally able to observe and understand the processes involved.

The studies were conducted as follows: subjects followed the Special Foods Diet described above and entered a VOC-free clean room and followed a protocol in the clean room that maintained the VOC-free environment. The VOC-free environment was possible because materials containing even extremely low levels of VOCs were not used. This was the only way to study the effects of VOCs at levels below the ability of analytical instruments to measure. Subjects lived in this VOC-free environment for from 4 weeks to 1 year.

In this pristine environment it was finally possible for this investigator to understand and clearly see the levels to which VOCs exposure needed to be reduced and why previously it had been only possible to achieve fluctuating symptom levels. It was here in VOC-free clean rooms that this investigator was finally able to observe and understand the processes involved.

The important results were unanticipated and were as follows:
1 Reactions to VOC exposures were superimposed and concurrent, not cumulative.
2 Symptom levels caused by an exposure did not increase when exposure levels increased.
3 Severity of symptoms did not increase when there were multiple exposures; however new, additional symptoms were often observed when subsequent exposures were from new VOC compositions.
4 Immune system response to a particular VOC (or VOC mixture) caused the same symptom array and severity of response consistently, regardless of whether exposures were open, single blind or double blind.
5 One extremely brief exposure at extremely low levels was all that was required to trigger a full immune system response.
6 One extremely brief exposure was equal to one sniff or one breath, approximately one second in duration. This short duration of exposure to VOCs was all that was required to trigger a full immune system response that could cause a reaction with symptom duration of a minimum of 4 days to 2 weeks typically, but could last as long as 2-3 months. This was possible to observe because this investigator was able to eliminate the exposure and wait until all symptoms had disappeared.
7 In addition to the short duration of exposure described in 6 above, exposure levels in the part per quadrillion level and below were sufficient to trigger a full response with strong return of symptoms lasting from 4 days to 2 weeks or more.
8 Subjects initially were frantic to maintain their previous exposure levels, and showed obvious signs of severe addiction and the associated withdrawal, cravings, and other behaviors. Autistic children resorted to spreading bodily fluids, accumulating sweat, urine, saliva and/or stool in wall crevices and doors where mold would grow, and if they were present, following tiny VOC plumes by scent location to their source (found in a few of the early clean rooms) and then taking ‘hits’. Children also used all kinds of manipulations, diversions and creative tactics to try to achieve VOC exposures without caregivers becoming suspicious. Non-autistic individuals experienced periods of intense feelings and symptoms, including but not limited to frustration, closed-in feelings, nervousness, jittery sensations, anxiety and the like.

9 In the span of 2-3 reaction cycles the symptoms, cravings and signs of addiction were eliminated as long as there were no exposures to VOCs during that time period.

With approximately half of the US population suffering from one or more chronic diseases, essentially every household is affected. The need is great for elimination of VOC exposures to the point that they are essentially not present in the environments in which people spend the bulk of their time.

The breakthrough research of applicant has shown that partial improvements are essentially meaningless.

Present Invention:

The primary purpose of the present invention is to describe methods for achieving the VOC reduction, sufficient to eliminate the chronic symptoms/conditions/diseases/disorders of millions, thereby saving lives, and saving billions of dollars in medical expenses, lost wages and lost productivity.

It was quickly determined that the following strategies, based upon the above described discoveries of applicant, would be of no benefit, would be essentially worthless and would be a great waste of money and effort, because they would not even partially eliminate chronic symptoms/conditions/diseases/disorders:

Non-Workable, Ineffective Strategy 1: Partially Reduce some Exposures.
Although at first partially reducing some exposures would seem to make sense at least as a start, it is a totally inappropriate and ineffective strategy. This strategy derives from the old ‘toxic effect’ strategy. Toxic effects (which chronic symptoms/conditions/diseases/disorders are NOT) occur at much higher exposure levels, generally the part per million to even as high as percent levels, and the levels are approximately a million times higher than the immune system triggered responses described in the instant invention. Toxic materials are taken into the body at such high levels that interfere directly with mechanical functions of organ systems, enzymes, and the ability of the body to uptake and utilize nutrients.
Standard toxic effects are generally dose dependent. This means that by dropping the intake levels by half, for example, there is generally a corresponding, significant reduction in the symptoms/effects caused by the substance.
Further for a standard toxic effect, it is relatively easy to drop exposure to a no-effect level. Generally, dropping exposures to one percent of the level that triggers a toxic effect, is sufficient to eliminate exposure-related effects. One percent reduction of a 1 part per million toxic effect is 10 part per billion. Most toxic standards are this level and higher.
For the average individual, total airborne VOC exposure is approximately 1 part per million. A 99% reduction would reduce the concentration in inhaled air to 10 parts per billion, as described. Unfortunately, this level is 10,000 to 10,000,000 times higher than the level at which the immune system is fully triggered, and therefore achieves essentially no effect.
The 99% reduction was the initial approach of applicant when developing a clean room approach. It was found that essentially only food-related symptoms were eliminated. As described above, it required virtual complete elimination of exposures to consistently eliminate symptoms of subjects suffering from chronic symptoms/conditions/diseases/disorders.

Non-Workable, Ineffective Strategy 2: Reduce VOC Exposures Dramatically in Some Areas, Leave Other Areas Unaddressed.
This non-workable strategy is another version of the theory that ‘something is better than nothing’ and again, it is totally ineffective.
As an example, in one case, exposure to plastic residues was eliminated from all of the sources listed above except one. Carefully purified water was stored for a few hours before use in a plastic storage unit. The subject's symptoms caused by exposure to plastics remained unchanged; these symptoms were not eliminated until the plastic storage container was discovered and changed to glass.
None of the following were capable of eliminating the chronic symptoms/conditions/diseases/disorders of the individuals studied:
   Leaving electrical cords in the room, eliminating everything else
   Washing clothes in tap water outside clean room, eliminating everything else
   Caregiver showering in unfiltered water outside clean room, eliminating everything else
   Storing food in plastic bowls, eliminating everything else

Non-Workable Ineffective Strategy 3: Install Air Filters
This is a non-workable strategy because of the low levels that are needed. Most of the time, use of air filters in lieu of modifying materials, products, furniture and the like is intended. The problem with this strategy is that exposure to VOCs generally occurs before the VOCs reach the filter, triggering the full scale reaction described previously. The instant invention shows clearly that filters in a room are useless in preventing the VOC exposures that trigger strong immune system responses.
In addition, air filters in the art are derived primarily of plastic housings and canisters, contain VOC impregnated resins, and pass over plastic fan components. The air filters themselves release VOCs to the air.
Finally, VOC removal to below the part per quadrillion level is beyond the capability of the filters of the art.

The invention of applicant successfully avoids the problems of the above unworkable strategies for elimination of VOC exposure, and describes effective strategies for eliminating chronic symptoms/conditions/diseases/disorders of many, by focusing on changing the environment to one in which the devastating immune system response is consistently avoided.

### SUMMARY OF THE INVENTION

### DETAILED DESCRIPTION OF THE INVENTION

In one embodiment of the invention Applicant follows the results of the research in which the Special Foods Diet was followed, and VOC exposures were reduced to the point that symptoms/conditions/diseases/disorders were consistently eliminated and the recovered state was maintained.

Although the instant invention describes effective treatment for a large variety of chronic symptoms/conditions/diseases/disorders, the instant embodiment treats the environment, the cause, rather than the individual. Unusual and unusually pure foods (the Special Foods Diet) are combined with extensive environmental modification to provide an environment in which the immune system is not triggered because VOC exposures are essentially absent.

In one embodiment the VOC exposures are reduced to the part per trillion level, in a preferred embodiment, VOC exposures are reduced to the part per quadrillion level, and in a still more preferred level VOC exposures are reduce to one thousand times below the part per quadrillion level.

In another embodiment specific techniques for reduction and elimination of volatile organic compounds are described, which the objective of obtaining the elimination of chronic symptoms/conditions/diseases/disorders.

A preferred embodiment involves fully eliminating all exposure areas simultaneously and maintaining this consistently. The advantage of this embodiment is that it is fully effective, the result is rapid elimination of the chronic symptoms/conditions/diseases/disorders, and rapid improvements in health, reduction of costs for medical treatments, and return to a productive life.

In one embodiment, safe/clean rooms that eliminate VOC exposure in combination with the Special Foods Diet are provided for treatment of chronic symptoms/conditions/diseases/disorders in long term care centers, recovery centers for the chronically ill, psychiatric centers, Alzheimer's care centers and the like.

In another embodiment, hospitals and medical treatment centers free of VOC exposures are provided so that medical interventions can be provided in the absence of VOC-triggered immune system attacks on the body. The benefits of this would be increased successes of medical treatments and procedures, increased survival rates, decreased recovery times, and reduced hospital costs.

In another embodiment, safe/clean rooms that eliminate VOC exposure in combination with the Special Foods Diet are provided for treatment of chronic symptoms/conditions/diseases/disorders in the home. The benefits of this are that individuals recovering from chronic symptoms/conditions/diseases/disorders are able to recover in their homes, in the company of family and friends. This is also less costly that recovering in a setting outside the home.

In another embodiment homes, apartments, hospitals, hotels, schools, offices, theaters, shopping centers, restaurants, churches, libraries, exercise and sports facilities and the like are provided that are free of VOC exposures. This embodiment extends the areas that are free of VOCs. This would be beneficial for individuals recovering from chronic symptoms/conditions/diseases/disorders. In addition, it would be strongly beneficial to individuals who have not yet progressed to the point of chronic disease, thus preventing essentially all chronic symptoms/conditions/diseases/disorders.

In yet another embodiment, cars, buses, planes, trucks and the like are provided that are free of VOC exposures. This embodiment also extends the areas that are free of VOCs. This would be beneficial for individuals recovering from chronic symptoms/conditions/diseases/disorders. In addition, it would be strongly beneficial to individuals who have not yet progressed to the point of chronic disease, thus preventing essentially all chronic symptoms/conditions/diseases/disorders.

A further embodiment of the instant invention is to provide a method of increasing the effectiveness of the human immune system comprising reducing the exposure of a human patient to VOCs by locating sources of VOCs to which the human patient is being exposed and reducing the VOC exposure by applying alternate materials and techniques including but not limited to those described in the examples below that eliminate the sources of VOC exposure, such as coating the VOC source with a material comprising glass or a glass precursor.

In a further embodiment of the instant invention a method of restoring health to a patient comprising eliminating from the patient's environment sources of exposure to volatile organic compounds, is provided.

In an additional embodiment is a method of preventing chronic disease in a human patient comprising reducing or eliminating the exposure of the patient to VOCs.

In order to provide clean rooms, homes, hospitals, schools, vehicles and the like that are free of exposure to VOCs, and provide the benefits described above, materials of composition must be changed from the current materials that emit moderate and high levels of VOCs. These are described herein in the embodiment, including the examples described below.

It has been found that the following products are inappropriate for individuals sensitive to volatile organic compounds: paints, synthetic clothing, plastics, pesticides, pressed wood furniture, composite wood furniture, soaps, detergents, perfumes, scented products, plastic polymers, synthetic resins, and all materials made from them.

It has further been found that volatile organic compounds represent inappropriate exposures and health hazards for everyone, especially for hypersensitive people showing adverse health effects including neurological symptoms, physical symptoms, psychological symptoms and/or effects, autism, chronic symptoms, chronic diseases, immune system activation, neuroimmune system effects, neuroautoimmune system impairment and dysfunction, chronic diseases caused by or triggered by the immune system, mood disorders, behavioral disorders, seizures, asthma, diabetes, obsessive/compulsive disorders, anxiety, arthritis, asthma, colic, congestion, diabetes, digestive upsets, irritable bowel syndrome, eczema, fatigue, migraine headaches, multiple sclerosis, and rashes, diarrhea, constipation, congestion, fatigue, muscle weakness, tension, and spasms, irritable bowel syndrome, swelling, anxiety, multiple chemical sensitivities, moderate to extensive and moderate to severe symptoms due to food allergies, sensitivities, and intolerances, bloating, pain, headaches, leaky gut, hyperactivity, sleeping difficulties, severe underweight, eating disorders, obsessive, compulsive disorders, panic attacks, sensory sensitivities, Alzheimer's disease, acid reflux, irritability, delayed motor skills, delayed social skills, autism, PDD, infantile spasms, and seizures.

Products useful in treating hypersensitivities are those that remove volatile organic compounds from the environment and from products used by and surrounding the hypersensitive individual.

Products disclosed herein are for the treatment of the above conditions, diseases and disorders.

### EXAMPLE 1

Tubing interior free of volatile organic compounds, non-bound, free reaction products and byproducts, partial polymerization products, incomplete reaction products, intermediates, raw materials, solvents used in manufacture, catalysts, and the like.

Tubing, pipes, hollow rods and other products for conveying or holding liquid, fluids, gaseous material streams, or solid particles, in which the interior is free of volatile organic compounds, non-bound, free reaction products and byproducts, partial polymerization products, incomplete reaction products, intermediates, raw materials, solvents used in manufacture, catalysts, and the like, provide an interior seal to the interior of the tubing, and is made as follows (letters below are for ease of reference):

a) apply a coating of glass, and/or a thin layer of glass, and/or glassy material, including and not limited to silica, silicon dioxide, silicon monoxide, borosilicate glasses, borate glasses, aluminoborates, aluminosilicates, soda-lime silicates, sodium silicates, phosphate glasses, transition metal glasses, rare earth glasses, simple silicates, complex silicates, germinates, tellurites, oxide glasses, boric oxide glasses, non-oxide glasses, chalcogenides, fluorides, metallic glasses, and combinations of the same,

b) in thicknesses or layers comprising: 1-20 micron-size layers, I to 100 monomolecular layers, several angstroms thick, ranging from one molecular layer to over 20 microns in thickness,

c) applied by: vapor deposition, vapor spraying, chemical bonding, chemical reaction, with and without heat, with vacuum including high vacuum and without vacuum, with pressure including high pressure and without pressure, and other techniques of the art,

d) for applying ultra thin layers to the interior surfaces of organic, polymer tubing and tubing combinations, ranging from flexible to non-flexible,

e) composed of the following polymers and polymer combinations: including tygon, polypropylene, acetate, acrylic, fiberglass, butyrate, phenolic, cellulose, fluorocarbon resins, vinyl, Teflon, chemfluor, cilran, polyester, copolyester, CPVC, Delrin, elastic, epoxy, ETFE, EVA, FEP, fluoropolymer, Garolite, rubber, gum. rubber, latex, hypalon, hydronic, inconel, Kevlar, Kynar, Masterkleer, Neoprene, Nitrile, norprene, nylon, PEEK, PETG, PEX, PFA, pharmed, plastic, pitot, polyethylene, polyimide, polyolefin, polypropylene, polyurethane, polyvinyl chloride, PTFE, PVC, PVDF, Santoprene, SBR, silicone, silicone rubber, surgical, Tefzel, thermoplastic elastomer, THV, Tygothane, ultrahigh molecular weight polyethylene, urethane, very high molecular weight polyethylese, Viton, organometallic polymers, including and not limited to multiple resins and polymers and combinations of resins and polymers,

f) of many different grades, ranging from coarse, technical, standard, reagent, pure, high purity, ultrapure, to the highest purity,

g) wherein the inner surface material of (a) is sufficient to seal, prevent completely or partially, preferably completely, migration, diffusion, passing, movement, progression, passage, vaporization, outgassing, and/or volatilization through the inner surface layer,

h) of organic compounds contained in the polymer tubing and tubing combinations, ranging from flexible to non-flexible,

i) said compounds including and not limited to volatile compounds, low molecular weight compounds, compounds of carbon, hydrogen, in linear or cyclic configurations, saturated or unsaturated having carbon atoms ranging from 1 to 100, preferably 1-60, more preferably 1-30, compounds of carbon, hydrogen, and oxygen in linear or cyclic configurations, saturated or unsaturated having carbon atoms ranging from 1 to 100, preferably 1-60, more preferably 1-30, compounds of carbon, hydrogen, oxygen, and chlorine in linear or cyclic configurations, saturated or unsaturated having carbon atoms ranging from 1 to 100, preferably 1-60, more preferably 1-30, and said described compounds also including and not limited to other halogens, metallic atoms, non-metallic atoms, rare earths, and combinations of same, including volatile organic compounds, non-bound, free reaction products and byproducts, partial polymerization products, incomplete reaction products, intermediates, raw materials, solvents used in manufacture, catalysts, and the like

j) in single compounds, or as complex mixtures ranging from one molecule to 100 molecules, to 1000 molecules, to 10,000 molecules, to 70,000, to 100,000 molecules in complex, static or dynamic mixtures,

k) that would otherwise migrate in said tubing, through said tubing, from said tubing, and/or into the interior of said tubing,

l) causing harmful effects, including and not limited to: adverse health effects, neurological symptoms, physical symptoms, psychological symptoms and/or effects, autism, chronic symptoms, chronic diseases, immune system activation, neuroimmune system effects, neuroautoimmune system impairment and dysfunction, chronic diseases caused by or triggered by the immune system, mood disorders, behavioral disorders, seizures, asthma, eczema, diabetes, obsessive/compulsive disorders, and genetic disorders including Angelman's syndrome, pemphigus vulgaris, porphyria, many others.

How to use: the product produced by the above method is used in hospitals, medical centers, clinics, nursing homes, care centers, convalescent facilities, and anywhere tubing is used to convey air, gasses, fluids, including body fluids to or from the body. The product is also for use residential, commercial and industrial as well as the above to eliminate VOC contamination from water pipes. Instant invention intended for tubing and piping and the like for any desired fluid conveyance, especially in instances where there is eventual human contact or uptake into the body. The interior seal prevents the harmful substances present in the tubing from contacting any part of the body of a person, and thus eliminate, avoid, halt the exposure to harmful substances described above, and the resultant adverse, harmful effects that occur when these air, gasses, fluids, including body fluids pass through unprotected tubing. The tubing has the advantage of otherwise maintaining the desirable properties of the above described tubing including but not limited to, size, shape, flexibility, resistance to breakage, reduced weight, ease of use. Provides for faster recovery from illness, surgeries and medical procedures, since adverse effects on the immune system from exposure to the unsealed tubing are avoided, and no adverse effects on enzyme function can occur from exposure to the unsealed tubing. The product maintains the interior seal since the thinness of the internal layer can be sufficiently thin to allow the inner layer to become flexible.

Also for use in treatment of hypersensitive individuals having at least one symptom or condition selected from the group consisting of autism, anxiety, arthritis, asthma, colic, congestion, diabetes, digestive upsets, irritable bowel syndrome, eczema, fatigue, migraine headaches, multiple sclerosis, seizures and rashes, Diarrhea, constipation, congestion, eczema, asthma, fatigue, muscle weakness, tension, and spasms, irritable bowel syndrome, swelling, anxiety, multiple chemical sensitivities, moderate to extensive and moderate to severe symptoms due to food allergies, sensitivities, and intolerances, bloating, pain, headaches, leaky gut, hyperactivity, sleeping difficulties, severe underweight, eating disorders, obsessive, compulsive disorders, panic attacks, sensory sensitivities, Alzheimer's disease, acid reflux, irritability, delayed motor skills, delayed social skills, autism, PDD, infantile spasms, seizures.

The product produced by the above method is tubing, pipes and the like free of volatile organic compounds, free of non-bound organic compounds, free of reaction products and byproducts, partial polymerization products, incomplete reaction products, intermediates, raw materials, solvents used in manufacture, catalysts, and the like.

### EXAMPLE 2

Tubing, pipes and the like including various shapes, free of volatile organic compounds, free of non-bound organic compounds, free of reaction products and byproducts, partial polymerization products, incomplete reaction products, intermediates, raw materials, solvents used in manufacture, catalysts, and the like.

In a derivitive approach from Example 1, the products are made by the process in which a sufficient amount of the glassy material of a) is mixed with said above described polymers and polymer combinations to form a matrix, a mass in which the glassy materials surround, incorporate and/or combine with the above described polymers and polymer combinations of e), applied by (c) with and without heat, pressure, radiation, microwave radiation, light, gasses and liquids to cure and form a mass including an infused mass of grades (f), which is then extruded, shaped, formed, milled and the like to form the desired tubing or shapes. Said mixture is sufficient in glassy material (a) to seal, prevent completely or partially, preferably completely, migration, diffusion, passing, movement, progression, passage, vaporization, outgassing, and/or volatilization from the mass, while still providing the desirable properties of the polymer or polymer combination. The glassy material of a) forms a thin, coating, layer of thickness (b) around polymer and polymer combinations (e) particles throughout the matrix, accomplishing throughout the mass by (c), the effect achieved in Example 1 above in the interior. The material is then extruded, milled, shaped into the desired shape according to the conventions of the art.

How to use: the product produced by the above method is used in hospitals, medical centers, clinics, nursing homes, care centers, convalescent facilities, and anywhere tubing is used to convey air, gasses, fluids, including body fluids to or from the body. The product is also for use residential, commercial and industrial as well as the above to eliminate VOC contamination from water pipes. Instant invention intended for tubing and piping and the like for any desired fluid conveyance, especially in instances where there is eventual human contact or uptake into the body. The interior seal prevents the harmful substances present in the tubing from contacting any part of the body of a person, and thus eliminate, avoid, halt the exposure to harmful substances described above, and the resultant adverse, harmful effects that occur when these air, gasses, fluids, including body fluids pass through unprotected tubing. The tubing has the advantage of otherwise maintaining the desirable properties of the above described tubing including but not limited to, size, shape, flexibility, resistance to breakage, reduced weight, ease of use. Provides for faster recovery from illness, surgeries and medical procedures, since adverse effects on the immune system from exposure to the unsealed tubing are avoided, and no adverse effects on enzyme function can occur from exposure to the unsealed tubing. The product maintains the interior seal since the thinness of the internal layer can be sufficiently thin to allow the inner layer to become flexible. Also for use in treatment of hypersensitive individuals having at least one symptom or condition selected from the group consisting of autism, anxiety, arthritis, asthma, colic, congestion, diabetes, digestive upsets, irritable bowel syndrome, eczema, fatigue, migraine headaches, multiple sclerosis, seizures and rashes, Diarrhea, constipation, congestion, eczema, asthma, fatigue, muscle weakness, tension, and spasms, irritable bowel syndrome, swelling, anxiety, multiple chemical sensitivities, moderate to extensive and moderate to severe symptoms due to food allergies, sensitivities, and intolerances, bloating, pain, headaches, leaky gut, hyperactivity, sleeping difficulties, severe underweight, eating disorders, obsessive, compulsive disorders, panic attacks, sensory sensitivities, Alzheimer's disease, acid reflux, irritability, delayed motor skills, delayed social skills, autism, PDD, infantile spasms, seizures

In addition, the product can be prepared in many shapes, for uses in toys, furniture, boxes, plates, dishes, structural materials and the like.

The product produced is at least partially free to completely free of volatile organic compounds, free of non-bound organic compounds, free of reaction products and byproducts, partial polymerization products, incomplete reaction products, intermediates, raw materials, solvents used in manufacture, catalysts, and the like.

### EXAMPLE 3

Provide an exterior seal to the exterior of tubing by the method described in Example 1 in its entirety, with the exception that the word ‘exterior’ be substituted for the word ‘interior’.

### EXAMPLE 4

Provide an interior and exterior seal to tubing by the method described in Example 1 in its entirety, with the addition that the words ‘and exterior’ be added after each word ‘interior’.

### EXAMPLE 5

provide a coating for plastic bottles, jars, containers, lids, glasses, dishes, food storage containers, and the like, so for interior and/or exterior they will be free of volatile organic compounds, non-bound, and free of reaction products and byproducts, partial polymerization products, incomplete reaction products, intermediates, raw materials, solvents used in manufacture, catalysts, and the like

Plastic bottles, jars, containers, lids, glasses, dishes, food storage containers, and the like with interior and/or exterior free of volatile organic compounds, non-bound, free reaction products and byproducts, partial polymerization products, incomplete reaction products, intermediates, raw materials, solvents used in manufacture, catalysts, and the like are made as follows (Letters below are for ease of reference):

To provide an interior and/or exterior seal to objects including plastic bottles, jars, containers, lids, glasses, dishes, food storage containers, and the like:

a) apply a coating of glass, and/or a thin layer of glass, and/or glassy material, including and not limited to silica, silicon dioxide, silicon monoxide, borosilicate glasses, borate glasses, aluminoborates, aluminosilicates, soda-lime silicates, sodium silicates, phosphate glasses, transition metal glasses, rare earth glasses, simple silicates, complex silicates, germinates, tellurites, oxide glasses, boric oxide glasses, non-oxide glasses, chalcogenides, fluorides, metallic glasses, and combinations of the same,

b) in thicknesses or layers comprising: 1-20 micron-size layers, 1 to 100 monomolecular layers, several angstroms thick, ranging from one molecular layer to over 20 microns in thickness,

c) applied by: vapor deposition, vapor spraying, chemical bonding, chemical reaction, with and without heat, with vacuum including high vacuum and without vacuum, with pressure including high pressure and without pressure, and other techniques of the art

d) for applying ultra thin layers to the interior and/or exterior surfaces of objects of organic, polymer and polymer combinations including plastic bottles, jars, containers, lids, glasses, dishes, food storage containers, and the like, ranging from flexible to non-flexible,

e) composed of the following polymers and polymer combinations: including tygon, polypropylene, acetate, acrylic, fiberglass, butyrate, phenolic, cellulose, fluorocarbon resins, vinyl, Teflon, chemfluor, cilran, polyester, copolyester, CPVC, Delrin, elastic, epoxy, ETFE, EVA, FEP, fluoropolymer, Garolite, rubber, gum rubber, latex, hypalon, hydronic, inconel, Kevlar, Kynar, Masterkleer, Neoprene, Nitrile, norprene, nylon, PEEK, PETG, PEX, PFA, pharmed, plastic, pitot, polyethylene, polyimide, polyolefin, polypropylene, polyurethane, polyvinyl chloride, PTFE, PVC, PVDF, Santoprene, SBR, silicone, silicone rubber, surgical, Tefzel, thermoplastic elastomer, THV, Tygothane, ultrahigh molecular weight polyethylene, urethane, very high molecular weight polyethylese, Viton, organometallic polymers, including and not limited to multiple resins and polymers and combinations of resins and polymers,

f) of many different grades, ranging from coarse, technical, standard, reagent, pure, high purity, ultrapure, to the highest purity,

g) wherein the inner and/or outer surface material of (a) is sufficient to seal, prevent completely or partially, preferably completely, migration, diffusion, passing, movement, progression, passage, vaporization, outgassing, and/or volatilization through the inner and/or outer surface layer,

h) of organic compounds contained in the polymer tubing and tubing combinations, ranging from flexible to non-flexible,

i) said compounds including and not limited to volatile compounds, low molecular weight compounds, compounds of carbon, hydrogen, in linear or cyclic configurations, saturated or unsaturated having carbon atoms ranging from 1 to 100, preferably 1-60, more preferably 1-30, compounds of carbon, hydrogen, and oxygen in linear or cyclic configurations, saturated or unsaturated having carbon atoms ranging from I to 100, preferably 1-60, more preferably 1-30, compounds of carbon, hydrogen, oxygen, and chlorine in linear or cyclic configurations, saturated or unsaturated having carbon atoms ranging from I to 100, preferably 1-60, more preferably 1-30, and said described compounds also including and not limited to other halogens, metallic atoms, non-metallic atoms, rare earths, and combinations of same, including volatile organic compounds, non-bound, free reaction products and byproducts, partial polymerization products, incomplete reaction products, intermediates, raw materials, solvents used in manufacture, catalysts, and the like

j) in single compounds, or as complex mixtures ranging from one molecule to 100 molecules, to 1000 molecules, to 10,000 molecules, to 70,000, to 100,000 molecules in complex, static or dynamic mixtures,

k) that would otherwise migrate in said object, through said object, from said object, into the interior of said object, from the exterior of said object,

l) causing harmful effects, including and not limited to: adverse health effects, neurological symptoms, physical symptoms, psychological symptoms and/or effects, autism, chronic symptoms, chronic diseases, immune system activation, neuroimmune system effects, neuroautoimmune system impairment and dysfunction, chronic diseases caused by or triggered by the immune system, mood disorders, behavioral disorders, seizures, asthma, eczema, diabetes, obsessive/compulsive disorders, and genetic disorders including Angelman's syndrome, pemphigus vulgaris, porphyria, many others including those listed elsewhere in the instant invention.

The product produced by the method of Example 5 can be used in the food industry to coat interiors of plastic bottles, jars, containers and the like, to prevent harmful substances present in the plastic objects from contacting the food and thus eliminate, avoid, halt the exposure to harmful substances described above (L), and the resultant adverse, harmful effects that occur. The product produced by the above method can also be used in the beverage industry. The product produced by the above method can be used in any situation in which the light weight arid flexibility of plastic is desired while the harmful effects of plastic exposure need to be avoided, including applications in which there is ultimately contact directly or indirectly with humans.

Also for use in treatment of hypersensitive individuals having at least one symptom or condition selected from the group consisting of autism, anxiety, arthritis, asthma, colic, congestion, diabetes, digestive upsets, irritable bowel syndrome, eczema, fatigue, migraine headaches, multiple sclerosis, seizures and rashes, diarrhea, constipation, congestion, eczema, asthma, fatigue, muscle weakness, tension, and spasms, irritable bowel syndrome, swelling, anxiety, multiple chemical sensitivities, moderate to extensive and moderate to severe symptoms due to food allergies, sensitivities, and intolerances, bloating, pain, headaches, leaky gut, hyperactivity, sleeping difficulties, severe underweight, eating disorders, obsessive, compulsive disorders, panic attacks, sensory sensitivities, Alzheimer's disease, acid reflux, irritability, delayed motor skills, delayed social skills, autism, PDD, infantile spasms, seizures

The products, objects including plastic bottles, jars, containers, lids, glasses, dishes, food storage containers, and the like of Example 5 are preferably at least partially free (at least 90 % VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99 % VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999 % VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like.

### EXAMPLE 6

Objects including plastic bottles, jars, containers, lids, glasses, dishes, food storage containers, and the like free of volatile organic compounds, free of non-bound organic compounds, free of reaction products and byproducts, partial polymerization products, incomplete reaction products, intermediates, raw materials, solvents used in manufacture, catalysts, and the like, are made by the process of Example 2 in which said process is used herein to prepare objects including plastic bottles, jars, containers, lids, glasses, dishes, food storage containers, and the like free of volatile organic compounds, free of non-bound organic compounds, free of reaction products and byproducts, partial polymerization products, incomplete reaction products, intermediates, raw materials, solvents used in manufacture, catalysts, and the like.

How to use: the product produced by the above method can be used in the food industry to manufacture plastic bottles, jars, containers and the like, to prevent harmful substances present in the plastic objects from contacting the food and thus eliminate, avoid, halt the exposure to harmful substances described above (L), and the resultant adverse, harmful effects that occur. The product produced by the method of Example 6 can also be used in the beverage industry. The product produced by the above method can be used in any situation in which the light weight and flexibility of plastic is desired while the harmful effects of plastic exposure need to be avoided.

Also for use in treatment of hypersensitive individuals having at least one symptom or condition selected from the group consisting of autism, anxiety, arthritis, asthma, colic, congestion, diabetes, digestive upsets, irritable bowel syndrome, eczema, fatigue, migraine headaches, multiple sclerosis, seizures and rashes, diarrhea, constipation, congestion, eczema, asthma, fatigue, muscle weakness, tension, and spasms, irritable bowel syndrome, swelling, anxiety, multiple chemical sensitivities, moderate to extensive and moderate to severe symptoms due to food allergies, sensitivities, and intolerances, bloating, pain, headaches, leaky gut, hyperactivity, sleeping difficulties, severe underweight, eating disorders, obsessive, compulsive disorders, panic attacks, sensory sensitivities, Alzheimer's disease, acid reflux, irritability, delayed motor skills, delayed social skills, autism, PDD, infantile spasms, seizures

The products, objects including plastic bottles, jars, containers, lids, glasses, dishes, food storage containers, and the like produced by the method of Example 6 are preferably at least partially free (at least 90% VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like.

### EXAMPLE 7

The inventions of Examples 1-6 can be further enhanced by the action of treating with hydrogen peroxide, oxygen, nitrogen, excess quantities of air, and the like. In each of the Examples above, the added step of treating with excess air or powerful oxidizers is added. The primary feature, characteristic of the polymers and polymer combinations described above (e) that contributes to toxicity is the availability of small molecular weight substances in the polymer matrix to transition from the matrix of the polymer to the air, body fluids, or a nearby surface. This may be reduced significantly by accelerating the removal of these substances that are migrating in the matrix that are not bound by the polymer.

How to make: to the polymer matrix prior to extrusion, or to the product after extrusion, combine hydrogen peroxide to the mixture and mix briefly, or alternatively, bubble hydrogen peroxide gas through the mass, separately or in combination with treatment with hydrogen peroxide gas or liquid after extrusion or shaping of the object. Contact time is sufficiently long to contact and remove or reduce the levels of small molecules in the matrix of extruded or shaped product. Sufficient quantities of hydrogen peroxide can be added to provide usefulness even when cellulosic polymers are involved. This method may be used separately or in combinations with the methods of Examples 1-6. The peroxide accelerates the kinetics to favor the space outside the matrix, since it immediately removes or destroys the compounds as soon as they migrate from the polymer matrix. This method involves removal of small molecules preferably to at least the ppt level and lower, more preferably to the at least the ppq level and lower, and most preferably to below the part per quadrillion level.

Alternatively ozone may be used as described above. Alternatively, excess air, oxygen or nitrogen, may be mixed with the matrix prior to extrusion, or blown over the product after extrusion to accelerate the rate of release of volatile substances. Alternatively these may be used sequentially or simultaneously. Alternately these may be used alone or in combination with the processes of Examples 1-6.

How to use: The products produced by the above method can be used in any situation in which the light weight and flexibility of plastic is desired while the harmful effects of plastic exposure need to be avoided.

Products preferably at least partially free (at least 90% VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like are produced by the method of Example 7.

### EXAMPLE 8

Maintaining flexibility of plastic in absence of small molecules are prepared in a modification of the method described above in Example 7. How to make: Polymers (e) treated in Example 7 above until free of small molecules, volatile organic compounds, and the like, may exhibit a reduction or loss of flexibility. To solve this problem, the treated polymer obtained by the method described in Example 7, is pulverized until ultra fine, particle size small enough to pass through a screen of 400 mesh, preferably to pass through a screen of 800 mesh, more preferably through a screen of 1200 mesh or smaller, must be ultrafine. Although the polymer fragments thus obtained will be small, they will be larger than the size of the volatile organic compounds that have been removed, and will not be subject to volatilization. The fine powder thus obtained is added to the during the production process, after the matrix is formed. The process proceeds with VOC removal techniques above until the removal is achieved. Flexibility is now provided by the small fragments of the pulverized matrix that slip between the long chain polymer units, allowing them to slip back and forth and providing flexibility desired.

Alternatively, flexibility is provided by finely pulverized glass or glassy materials of (a) of the desired, above described particle size. Flexibility is now provided by the small fragments of the pulverized glass or glassy that slip between the long chain polymer units, allowing them to slip back and forth and providing flexibility desired. Most preferred is ultra fine particle size.

How to use: the product produced by the method of Example 8 can be used in uses typical of the art, replacing the loss of flexibility associated with removal of harmful volatile organic compounds from the product.

A product of the method of Example 8 is preferably at least partially free (at least 90% VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like, maintains desired properties of flexibility or improves the flexibility of products of Examples 2, 6, and 7.

### EXAMPLE 9

plastic food wrap. By vapor deposition silicon oxide, or silicon dioxide may be added to plastic materials, the instant invention involves treating both sides of the plastic with vapor deposition to form a plastic sheet with flexibility of plastic and full properties of glass. In addition there is the addition of thin layers of sodium silicate as an adhesive. 20-60% solutions of sodium silicate, preferably 40% sodium silicate are used, or alternatively are diluted 1:3, sodium silicate solution: water, to 1:10 ratio sodium silicate solution: water, or alternatively, a mixture of sodium silicate solution of the described concentrations is combined with a variety of other materials. The resultant mixture is pasted thinly onto the glass treated sheets, and allowed to dry spontaneously or drying is accelerated with heat guns, microwaves, ovens or other methods for adding heat or of drying. This mixture of sodium silicate and water provides a layer of sodium silicate that has flexible properties. The adhesive, sodium silicate, holds the object together well, allowing use in packaging. Also capable of maintaining a vacuum

How to use: use in food wrapping, packing sandwiches, making lunch bags, wrapping and sealing meats as a substitute for vacuum packing with plastic. Material thus produced my be used as a surface covering for walls, ceilings, floors, in building interiors, as vapor barriers, and the like and in other applications that prevent the release of VOCs.

A flexible sheet product suitable for including but not limited to: food wrapping, packing sandwiches, making lunch bags, wrapping and sealing meats, surface covering for walls, ceilings, floors, in building interiors, as vapor barriers, and the like and in other applications that prevent the release of VOCs and as a substitute for vacuum packing with plastic, and although VOCs are present in the plastic interior of the product, the surfaces are free of volatile organic compounds, free of non-bound organic compounds, free of reaction products and byproducts, partial polymerization products, incomplete reaction products, intermediates, raw materials, solvents used in manufacture, catalysts, and the like and yet the desired properties of flexibility are maintained.

A thin, flexible adhesive of sodium silicate capable of maintaining a vacuum is provided.

### EXAMPLE 10

sealant paint for vapor control.

How to make: overlapping disks of aluminum foil, particle size ranging from 0.5 in, 0.25 in, to 0.01 inch is stirred into a matrix of sodium silicate in a liquid form, approximately 40% solution of sodium silicate in water, preferably 20% solution of sodium silicate in water, more preferably 10% solution of sodium silicate in water, still more preferably 5% solution of sodium silicate in water, most preferably less than 5% solution of sodium silicate in water, to form a combination of foil and sodium silicate solution that is approximately equal parts foil and solution. The mixture is painted onto a wall slowly until the entire area is covered. Then allowed to dry, then a second coat is applied. Immediately following the second application, the solution is sponged thoroughly on the surface to remove excess sodium silicate. Sodium silicate solution provides adhesion, and ease of application and forming a glass seal. The foil also provides a vapor barrier, and also covers the sodium silicate, protecting and keeping the sodium silicate from crumbling, taking up moisture, and preventing deterioration. Provides a durable, long lasting seal. How to use: use as a sealing paint for walls, floors, ceilings, to seal away the fumes from materials of construction, permanently and tightly. Also use as a covering, coating for furniture. .

A sealant paint free of volatile organic compounds, for sealing walls, floors, ceilings, and objects to eliminate exposure to volatile organic compounds seeping from within walls, and the like ia produced.

### EXAMPLE 11

the paint of Example 11, to which is added calcium carbonate or calcium sulfate or other calcium or magnesium compounds. How to make: The paint sealant is applied as described in Example 10. While wet, the walls, still wet with the foil and excess sodium silicate solution, are coated with an excess quantity of calcium carbonate or calcium sulfate. The calcium reacts with the sodium silicate to form an insoluble silicocalcium matrix and provide a white color. Alternatively the powder can be tinted or powdered with any desired color or shade. Most preferred is tinting with mineral pigments.

A VOC sealant compound in a coating of calcium powder is added to the wet surface forming a silicocalcium reaction product that stabilizes the sodium silicate, prolongs the useful life of the coating, and provides a white color.

### EXAMPLE 12

hypoallergenic cotton. An improved cotton product is prepared having essentially eliminated small molecules, including small organic molecules. How to make: cotton is first wetted with a surfactant, and rinsed thoroughly, then sodium hydroxide scouring agent is added, with concentration between 10 and 20 percent and heating time and temperature according to the teachings of the art, or alternatively typical times and temperatures slightly increased by 1-10% to which solution sodium hexametaphosphate is added as a chelating agent. Then hydrogen peroxide in combination with sodium silicate is added to the bleaching step, followed by steam drying, and combing. Water for washes and rinses is purified with activated carbon for removal of chlorinated and nonchlorinated organic compounds. This produces an ultrapure cotton product free of impurities, not previously known. Steps of citric acid treating, gas fired drying, and coating with silicone are eliminated. Sodium hexametaphosphate replaced sodium polyacrylate and sodium gluconate as chelating agents, and sodium silicate replaced complex phosphonate as stabilizer during the bleaching process. How to use: this ultrapure cotton is free of natural waxes and terepinoids and the like, and also free of contaminants that would otherwise be added during processing. It is useful for individuals with the chronic symptoms/conditions/diseases/disorders listed herein since it eliminated VOC exposure from cotton cloth, fabric and all associated uses of same. It is also important as a means for preventing currently asymptomatic individuals from experiencing chronic symptoms/conditions/diseases/disorders.

The product produced by the above method is used in hospitals, medical centers, clinics, nursing homes, care centers, convalescent facilities, also clothing of all sorts and types, and anywhere cotton is used. The product would prevent the harmful substances present in commercial and natural cotton from contacting any part of the body of a person, and thus eliminate, avoid, halt the exposure to harmful substances described above, and their associated effects which are described elsewhere in this application.

Also for use in treatment of hypersensitive individuals having at least one symptom or condition selected from the group consisting of autism, anxiety, arthritis, asthma, colic, congestion, diabetes, digestive upsets, irritable bowel syndrome, eczema, fatigue, migraine headaches, multiple sclerosis, seizures and rashes, diarrhea, constipation, congestion, eczema, asthma, fatigue, muscle weakness, tension, and spasms, irritable bowel syndrome, swelling, anxiety, multiple chemical sensitivities, moderate to extensive and moderate to severe symptoms due to food allergies, sensitivities, and intolerances, bloating, pain, headaches, leaky gut, hyperactivity, sleeping difficulties, severe underweight, eating disorders, obsessive, compulsive disorders, panic attacks, sensory sensitivities, Alzheimer's disease, acid reflux, irritability, delayed motor skills, delayed social skills, autism, PDD, infantile spasms, seizures.

The cotton product thus produced is preferably at least partially free (at least 90% VOC free) of volatile organic compounds, free of non-bound organic compounds, and the like, more preferably are mostly free (at least 99% VOC free) of volatile organic compounds, of non-bound organic compounds, and the like, still more preferably virtually completely free (at least 99.999% VOC free) of volatile organic compounds, free of non-bound organic compounds, and the like, most preferably completely free of volatile organic compounds, free of non-bound organic compounds, and the like.

### EXAMPLE 13

The cotton product produced by the method of Example 12, is further used to prepare woven fabric from the fibers. Woven in looms free of VOC-contamination and without the addition of chemicals in the weaving process other than non-VOC containing substances, it provides clothing, garments, bedding, furnishing and furnishing covers and the like that are free of VOC contamination to preferably at least as low as the part per billion level, more preferably to at least as low as the part per trillion level, and most preferably to at least as low as the part per quadrillion level.

The cotton products made from the purified cotton of Example 12, including clothing, garments, gloves, bedding, socks, pillow case, mattress cover, blankets, comforter, underpants, reusable sanitary napkins, disposable ianitary napkins, tampons and shower curtains, furnishing and furnishing covers and the like are at least partially free (at least 90% VOC free) of volatile organic compounds, free of non-bound organic compounds, and the like, more preferably are mostly free (at least 99% VOC free) of volatile organic compounds, of non-bound organic compounds, and the like, still more preferably virtually completely free (at least 99.999% VOC free) of volatile organic compounds, free of non-bound organic compounds, and the like, most preferably completely free of volatile organic compounds, free of non-bound organic compounds, and the like.

### EXAMPLE 14

new cotton fabric from the cotton of Example 12. How to make: the cotton of Example 12 is converted to fabric by the method of hydroentangling, using carbon filtered or water otherwise purified to eliminate VOC presence. This produces a cloth that has not been contaminated by looms, machine oils and the like, and that has the advantage of eliminating the dyeing and finishing stages of processing, providing a one step treatment and purification step followed by a cloth production that stays free of contamination. How to use: this ultrapure cotton is free of natural waxes and terepinoids and the like, and also free of contaminants that would otherwise be added during processing and weaving. It is useful for individuals with severe chemical sensitivities, to avoid serious, adverse reactions.

Ultrapure cotton products including linens, clothing, and the like are at least partially free (at least 90% VOC free) of volatile organic compounds, free of non-bound organic compounds, and the like, more preferably are mostly free (at least 99% VOC free) of volatile organic compounds, of non-bound organic compounds, and the like, still more preferably virtually completely free (at least 99.999% VOC free) of volatile organic compounds, free of non-bound organic compounds, and the like, most preferably completely free of volatile organic compounds, free of non-bound organic compounds, and the like.

### EXAMPLE 15

Portable vapor deposition, to allow spraying walls. It is known in the art to provide a spraying or application method that consists of a chamber through which sheets of plastic may be passed in order to provide a process for continuous application of ultra thin layers of various silicon oxides and other oxides capable of forming glassy layers, including SiO₂ and SiO on the surface of the materials. How to make: The present invention provides the spraying method above applied as described in Example 1(d) in a smaller and portable form in which the spraying or application method is passed over the surface of a substrate, with the result that ultra thin layers of various silicon oxides and other oxides capable of forming glassy layers, including SiO₂ and SiO including those identified above in Example 1(a), are applied to the surface of the substrate, including those identified above in Example 1(e), thus forming a barrier of thickness described in Example 1(b), ranging from at least partial to complete barrier preventing migration of volatile organic compounds and other substances in the substrate from migrating out of the substrate, through the applied barrier and into the air space above the substrate, as described in Example 1(g-1) with the proviso that the reference to tubing is expanded to include: surfaces in general including cellulosic materials, wall paper, painted surfaces of walls, floors, ceilings, textured surfaces, woven surfaces, cloth, fabric, paper, polymers and polymer combinations described in Example 1(e), combinations with surfaces in general including cellulosic materials, wall paper, painted surfaces of walls, floors, ceilings, textured surfaces, woven surfaces, cloth, fabric, paper.

In an expansion of the invention, the invention is modified to the spraying method to be applied at a large number of angles, in order to provide coverage of surfaces that are curved, have small imperfections, and/or include a wide variety of uneven surfaces, and yet still provide complete coverage and protection.

How to use: the present invention is used to seal the walls, floors, ceilings, surfaces in general of homes, businesses and commercial enterprises and surfaces of commercially produced items, to reduce or eliminate the release to the air of fumes, particularly volatile organic compounds, that would otherwise be released to air (outgas) from these materials.

A product for portable coating application to many surfaces, providing a thin layer of SiO₂, SiO and the like to the chosen surface and providing at least a partial barrier that prevents the migration of volatile organic compounds and other substances from the treated surface is provided in Example 15.

### EXAMPLE 16

Portable curing of sodium silicate, to convert to water insoluble form after application. Sodium silicate is a soluble form of glass that dries to a layer of glass, but has the disadvantage of being water soluble. Upon exposure to the air, it gradually becomes cloudy, and begins to dry and flake off as it absorbs moisture from the air. It has been found that heat applied in the correct amount of time can cause sodium silicate to polymerize and become water insoluble. The purpose of this invention is to provide a method by which the sodium silicate, once applied to various, surfaces including walls, floors, ceilings and the like, can be applied as a liquid and form the complete coverage of a wide variety of surfaces and shapes, taking advantage of the properties of sodium silicate as a liquid, and then applying a further treatment method to convert the sodium silicate, now applied in thin layers, to a water insoluble material having fully the properties of glass, thus providing thin layers of glass as a barrier to fumes to a wide variety of substrates. Sodium silicate polymerization may be accomplished by heat, microwave treatment, radiant heat treatment.

How to make: the instant invention is a method for applying energy to sodium silicate treated surfaces, said energy sources either singly or in combination provides energy from microwaves, infrared heat, incandescent heat, radiant heat, heat from electrical resistance, and a wide variety of other energy sources, in a portable form that can be moved over a sodium silicate treated surface, converting the sodium silicate to at least partially insoluble form, without causing significant deterioration or scorching of the treated surface, and providing an at least partial barrier against the migration of substances in the substrate beneath the sodium silicate layer from migrating through the barrier. How to use: apply invention as wand, hand held device, or other configuration to the sodium silicate treated surface, which is either completely or partially dried, moving over the sodium silicate treated surface at pace appropriate to allow the sodium silicate treated surface to convert to polymerized form and become a water insoluble and impervious barrier. Useful also for other substances capable of forming glassy materials, including those listed in Example 1(a), surface can include and not be limited to those listed and all possible combinations of surfaces in general including cellulosic materials, wall paper, painted surfaces of walls, floors, ceilings, textured surfaces, woven surfaces, cloth, fabric, paper, polymers and polymer combinations described in Example I (e), combinations with surfaces in general including cellulosic materials, wall paper, painted surfaces of walls, floors, ceilings, textured surfaces, woven surfaces, cloth, fabric, paper.

A wand, hand held or other device for converting sodium silicate or other materials capable of forming glassy layers, to an insoluble form without causing scorching or other damage to the underlying surfaced, thus forms a barrier that is capable of at least partially free (preventing at least 90% VOC migration) of VOCs, of non-bound organic compounds, and the like, more preferably are mostly free (preventing at least 99% VOC migration) of volatile organic compounds, of non-bound organic compounds, and the like, still more preferably virtually completely free (preventing at least 99.999% VOC migration) of volatile organic compounds, of non-bound organic compounds, and the like, most preferably completely free of migrating volatile organic compounds, of non-bound organic compounds, and the like, through the treated layer or surface.

### EXAMPLE 17

Treat walls with plaster of Paris or other suitable materials, then apply sodium silicate, let react to form stable bond and seal. How to make: a method for providing sodium silicate in an impervious form, that is stable, will not disintegrate, crack and peel, and will maintain an impervious barrier preventing or at least partially preventing the migration of volatile organic compounds and other substances described elsewhere in this application, through the sodium silicate barrier.

A layer of sodium silicate or other substance capable of forming glassy materials, including those listed in Example 1(a) is applied to a selected surface, said surfaces described above in the application, including but not limited to walls, floors, ceilings, frames, baseboards, various objects and the like, surface can include and not be limited to those listed and all possible combinations of surfaces in general including cellulosic materials, wall paper, painted surfaces of walls, floors, ceilings, textured surfaces, woven surfaces, cloth, fabric, paper, polymers and polymer combinations described in Example 1(e), combinations with surfaces in general including cellulosic materials, wall paper, painted surfaces of walls, floors, ceilings, textured surfaces, woven surfaces, cloth, fabric, paper. A layer of sodium silicate or other substance described above is applied to said surface and allowed to dry, as soon as the layer is suitably dry, and before significant deterioration can begin, a layer of sodium silicate or other suitable material as listed above is applied as a second coating. To this second coating, and while still wet, a dry layer of a powder containing a calcium compound is applied to the wet layer. The powder is pressed into the wet sodium silicate layer. The calcium in the dry material, reacts with the moist sodium silicate forming calcium silicate compounds that are stable and water insoluble. The result is a sodium silicate seal under a silicocalcium layer or similar silico layer that is impervious to water. This layer also protects the sodium silicate layer and prevents deterioration and peeling of the layer. Thus is formed a layer to which layers containing plaster of Paris, taping and bedding compounds, white wash, titanium dioxide, combinations containing iron oxides and various mineral pigments and other suitable materials can be applied.

Alternately, a single layer of sodium silicate can be used, following the method above, with the proviso that the calcium or other compound powder be applied to the first sodium silicate layer while still wet. Alternately, the dry powder can be applied as a layer first, dry powder to include plaster of Paris, calcium carbonate, calcium sulfate, and a wide variety of other calcium containing compounds including mixtures containing calcium. For application, said powder can be mixed with water or other suitable substances to thin to the point that it can be applied to a selected surface and allowed to dry. Sodium silicate or other suitable materials described above can then be added to this material thus providing a sodium silicate layer that is applied to a calcium-containing substrate or other suitable substrate, thus providing a site for reation to occur to form the desired impervious barrier. In this method, the sodium silicate or other suitable material must be applied thinly so that the entire layer will be available for reaction, and yet still provide the complete seal desired.

How to use: for application in which at least partial protection from outgassing of volatile organic compounds and other materials is desired, method avoids the need for polymerizing reaction of sodium silicate.

A method for treating surfaces with alternating layers of sodium silicate or other suitable materials and calcium containing layers or layers of other suitable materials or combinations, to provide reactions between the two layers, forming an impermeable barrier as a result of reaction between the two layers, provides a final layer, at least partially impervious to migration of outgassing substances from the underlying substrate, and providing a stable, water impervious outer surface. Thus preventing at least partial (preventing at least 90% VOC migration) of VOCs, of non-bound organic compounds, and the like, more preferably preventing at least 99% migration of volatile organic compounds, of non-bound organic compounds, and the like, still more preferably virtually completely free and preventing at least 99.999% migration of volatile organic compounds, of non-bound organic compounds, and the like, most preferably providing a surface completely free of migrating volatile organic compounds, of non-bound organic compounds, and the like.

### EXAMPLE 18

Microsphere of metal. The method of Example 17, above to which is added microspheres of metal to provide an additional barrier to migrating outgassing substances from the underlying substrate, and to provide a variety of color options. Microspheres may be added to either the sodium silicate layer, calcium or other suitable substance layer, or to both. Particle size is 80 mesh or smaller, preferable 200 mesh or smaller, preferably 400 mesh or smaller, still more preferably 800 mesh, still more preferably 1400 mesh, still more preferably 1 micron or less, still more preferably 0.01 microns or less in size. How to make: to the layers described in item 4 above, add microspheres to the material prior to applying the layer. Microspheres may be added to either the sodium silicate layer, calcium or other suitable substance layer, or to both. Particle size is 80 mesh or smaller, preferably 200 mesh or smaller, preferably 400 mesh or smaller, still more preferably 800 mesh, still more preferably 1400 mesh, still more preferably 1 micron or less, still more preferably 0.01 microns or less in size. How to use: for application in which at least partial protection from outgassing of volatile organic compounds and other materials is desired, method avoids the need for polymerizing reaction of sodium silicate.

A method for treating surfaces with alternating layers of sodium silicate or other suitable materials and calcium containing layers or layers of other suitable materials or combinations, to which has been added microspheres of metal or glass or glassy material composition to one or both layers, to provide an added barrier against migration of substances from the underlying substrate, providing color, and still provide reactions between the two layers, forming an impermeable barrier as a result of reaction between the two layers, providing a final layer, at least partially impervious to migration of outgassing substances from the underlying substrate, and providing a stable, water impervious outer surface. Thus preventing at least partial (preventing at least 90% VOC migration) of VOCs, of non-bound organic compounds, and the like, more preferably preventing at least 99% migration of volatile organic compounds, of non-bound organic compounds, and the like, still more preferably virtually completely free and preventing at least 99.999% migration of volatile organic compounds, of non-bound organic compounds, and the like, most preferably providing a surface completely free of migrating volatile organic compounds, of non-bound organic compounds, and the like.

### EXAMPLE 19

Portable furniture sprayer, for finishes on furniture. For furniture it is desirable to be able to treat the furniture with a finish and provide a stain that provides a desired color and texture. The wood itself contains volatile components and semivolatile components that must be protected from migration. In addition the materials used to provide coating, texture, tint and the like contain great amounts of undesirable materials. While new process changes can eliminate many of these problems, the furniture and furnishings already in existence must be modified or treated in a way that reduces or eliminates the problem of outgassing substances. How to make: in the present method, a method of spraying is provided that applies a thin layer of sodium silicate over the surface of the finished or unfinished furniture or objects including solid wood or composite materials. Once applied, the sodium silicate while wet is rubbed into the pores of the wood. The purpose is to enter and fill the spaces in the wood. The spaces will provide some protect for the wood and greatly slow deterioration, while preventing the migration of volatile organic compounds and other substances identified elsewhere in this application from the furniture, item or object. Alternating applications of and rubbing, as many as desired are provided in the instant invention. Finally a layer of warm softened (by heat) or melted beeswax is rubbed over the furniture, object, or item. This provides a final seal. Although the beeswax may be applied after the sodium silicate has dried, it is more preferably applied to the surface still moist from the final application of sodium silicate. This allows a combination of beeswax with the sodium silicate layer, forming an organosilicate layer. A second, final beeswax layer is also provided in the invention.

How to use: a portable sprayer is used to apply at close range, including point blank, sodium silicate into the pores of finished or unfinished wood products under pressures of up to and including 2800 psi, and including pressures of up to 500 psi, forcing the sodium silicate into the pores of the wood. This is combined with rubbing, and included cycles of alternating pressure application and rubbing, including one cycle, two cycles of spraying and rubbing, three cycles of spraying, and increasing to include 10 cycles of spraying and rubbing. This is followed by an application of beeswax to the surface with may be dry or preferably wet. The warm or melted beeswax is rubbed over the wet (preferably) or dry sodium silicate treated surface, in two layers, providing a sealed, protected surface that at least partially prevents the migration of volatile organic compounds and other substances described elsewhere in this application.

A portable sprayer for contacting treated and untreated surfaces of wood and other composition furniture, objects or items, forcing sodium silicate into the pores at high pressures, rubbing the sodium silicate into the pores in alternating cycles, and finally coating with layers of beeswax. Sealing the sodium silicate into the wood and providing at least partial protection from outgassing volatile organic compounds and other substances while providing protection for the sodium silicate and thus prolonging the effectiveness of the treatment, thus preventing at least partial (preventing at least 90% VOC migration) of VOCs, of non-bound organic compounds, and the like, more preferably preventing at least 99% migration of volatile organic compounds, of non-bound organic compounds, and the like, still more preferably virtually completely free and preventing at least 99.999% migration of volatile organic compounds, of non-bound organic compounds, and the like, most preferably providing a surface completely free of migrating volatile organic compounds, of non-bound organic compounds, and the like.

### EXAMPLE 20

Organic base paint: flour, titanium, calcium carbonate, or the like, binder of oil of the day, mineral pigments. A paint that is free of organic materials, and thus outgassing substances. How to make: paint requires a binder, a vehicle and a filler. Sometimes they can be interchangeable. Or one material provide more than one function. In the present invention water or H₂O₂ is provided as the vehicle, hypoallergenic oils treated to remove volatile organic compounds and other undesirable substances are used as binders, and unusual flours also treated to remove volatile organic compounds and other substances identified elsewhere in the application, as well as mineral based substances including but not limited to whiting, calcium carbonate, calcium sulphate, calcium silicate compounds, iron oxide compounds, to provide coverage and color to the painted surface. This paint has the advantage of avoiding everything associated with petroleum products or petroleum derived products, which contain many volatile organic compounds and moderately volatile organic compounds. Flour from vegetable sources is treated variously to remove volatile organic compound fractions by methods including but not limited to, passing through large excesses of air, nitrogen, oxygen, or passing through or treating with H₂O₂, or O₃. if treated with a liquid, said flour is combined with the liquid in a slurry, stirred for 5-10 minutes, then spread thinly and allowed to dry, and repulverized if necessary with particle size reduction techniques of the art. Alternatively may be treated in larger particle sizes with appropriate adjustments allowed for time for penetration. Oils used as binders are first treated with peroxide, ozone, or by carbon filtration to remove volatile organic compounds moderately volatile organic compounds, and the like and other compounds described elsewhere in the present application. Cooked flour can also serve as a binder, can be cooked, made into paste, and combined, or can be cooked, dried and pulverized to reconstitute when paint is made.

1) Combine: flour from vegetable sources, and/or flour from biologic sources, both treated as described in the paragraph above to remove volatile organic compounds and other undesirable substances described elsewhere, and/or flour from mineral sources, treated as needed to remove volatile organic compounds and other undesirable substances described elsewhere, with oil treated as described to remove or reduce volatile organic compounds and other undesirable substances described elsewhere, and/or water to provide a paint product.

2) Alternatively combine flour from vegetable sources, flour from biologic sources, both treated as described in the paragraph above to remove volatile organic compounds and other undesirable substances described elsewhere, and/or flour from mineral sources, treated as needed to remove volatile organic compounds and other undesirable substances described elsewhere, with cooked flour from vegetable sources, cooked flour from biologic sources, both treated as described in the paragraph above to remove volatile organic compounds and other undesirable substances described elsewhere, and/or flour from mineral sources, treated as needed to remove volatile organic compounds and other undesirable substances described elsewhere, with water or peroxide, at room temperature or heated, and non cooked flours from biological or mineral sources. To this may be added as desired oil that has been treated to remove volatile organic compounds and other undesirable substances described elsewhere.

How to use: apply paint to desired surfaces including walls, ceilings, floors, and objects.

A paint that is at least partially to completely free of volatile organic compounds and moderately volatile organic compounds, preferably at least partially free (at least 90% VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like

An additional product is food stuffs and edible products of all kinds including flour that is at least partially to completely free of volatile organic compounds and moderately volatile organic compounds. Thus the food of example 20 is at least partially free (at least 90% VOC free) of VOCs, of non-bound organic compounds, and the like, more preferably at least 99% free of volatile organic compounds, of non-bound organic compounds, and the like, still more preferably virtually completely free and at least 99.999% free of volatile organic compounds, of non-bound organic compounds, and the like, most preferably providing a product that is completely free of volatile organic compounds, of non-bound organic compounds, and the like.

### EXAMPLE 21

Dual nozzle approach that impacts microstreams at high pressure onto each other inside the heat or microwave radiation to the nozzles tool. High energy level, sufficient to convert sodium silicate from water soluble to insoluble. How to make: provide a nozzle suitable for impacting opposing streams of sodium silicate solution or other materials capable of forming glassy layers, to impact on each other at high velocity and high force, sufficiently high to provide the energy for converting sodium silicate or other compounds in the streams to insoluble form. Said insoluble form delivered to the intended surface as the change to insoluble form occurs, thus providing an instantly insoluble layer. Thus providing the protection from migrating volatile organic compounds and the like described elsewhere. In an additional embodiment, the above nozzle as described that has the added feature of impacting the streams against each other and simultaneously against a very hard structure on the inside surface of the nozzle, thus increasing the energy of the impact. Streams provided to the nozzle have high pressure, of 100 psi and greater, 500 psi and greater, 1000 psi and greater, 5000 psi and greater, still more preferred 10000 psi and greater, still more preferred 20000 psi and greater, increasing to and including 50000 and 100000 psi. In an additional embodiment, the nozzle also provides increased temperature, and the streams provided have increased temperature. In an additional embodiment, the nozzle is also modified to provide microwave energy to the nozzle reaction chamber.

How to use: the nozzle of the instant invention provides instant conversion of sodium silicate or other included materials to a finely divided particle size of insoluble sodium silicate or other included materials exiting from the nozzle and completing the transformation as the spray impacts with the treated surface, thus providing the desired product coverage with an impermeable barrier to volatile organic compounds and the like described elsewhere in this application.

A nozzle or other reaction chamber device for converting sodium silicate to insoluble form as it deposits onto the surface being treated is described.

### EXAMPLE 22

Clothing, peroxide treatment to allow people to wear their clothes. How to make: clothing, including cotton, but also including clothing containing synthetic and natural fibers of many types including those listed in Example 1(e) above, contain volatile organic compounds including those listed in Example 1 (i) above, that needs to be removed prior to use by the consumer. These compounds for removal are some inherent in the material, and others added as a result of manufacturing, treating, dying and finishing and other steps. These compounds may be removed by added final steps of washing in peroxide solution with suitable temperature and holding times, and/or by exposure to H₂O₂ and/or O₃ gasses in a holding chamber. How to use: addition of H₂O₂ to vats containing clothing, fabric and the like, to treat and remove the described volatile organic compounds and the like. Passing clothing, fabric and the like through water filled vats through which is bubbled O₃, spraying clothing, fabric and the like with H₂O₂ and/or O₃, letting set, then washing and rinsing. Chambers filled with H₂O₂ gas or O₃ gas, through which clothing, fabric and the like are passed.

A treatment for clothing, fabric and the like, which causes the removal of volatile organic compounds including those listed in Example 1(i) above, that need to be removed prior to use by the consumer. The clothing product prepared by the process of said treatment for clothing, fabric and the like, which causes the removal of volatile organic compounds including those listed in Example 1(i) above, that needs to be removed prior to use by the consumer. Thus providing a treated clothing, fabric and the like that is at least partially free (at least 90% VOC free) of volatile organic compounds, free of non-bound organic compounds, and the like, more preferably are mostly free (at least 99% VOC free) of volatile organic compounds, of non-bound organic compounds, and the like, still more preferably virtually completely free (at least 99.999% VOC free) of volatile organic compounds, free of non-bound organic compounds, and the like, most preferably completely free of volatile organic compounds, free of non-bound organic compounds, and the like. To which may be added to the above treatment to which is added the step of boiling, either simultaneously or sequentially of sufficient time to increase or accelerate the rate of removal of volatile organic compounds including those listed in Example 1(i) above.

### EXAMPLE 23

Books: printing: addition of final step to printing, to include 1) extra heat rollers for drying and driving off fumes, 2) vapor deposition of silicon oxide or other oxide capable of forming glassy materials, 3) ozone treatment, peroxide treatment, 4) coating vinyl binding with SiO₂ for glossy, sealed finish. 24 are described previously above and incorporated here. How to make: a process for eliminating the print and ink related fumes from books, that includes adding a set of steps to the end of the printing or binding process, step to be selected as appropriate for the part of the process involved. Process involves either providing an ultra thin coating that will seal volatile organic compounds including those listed in Example 1(i) above and trap them in the substrate, or provides a method to remove volatile organic compounds including those listed in Example 1(i) above through heat treatment and/or ozone or peroxide treatment. Selection to be made based on the intended use and on the desired degree of protection, and the type of inks involved. How to use: use by treating printed paper and other printed materials, bindings, covers and the like, using combinations of heat, coatings with SiO₂ SiO and other suitable materials, treatment with H₂O₂ and O₃ and related materials to achieve the desired reduction in levels of volatile organic compounds and other materials capable of migrating from the printed materials, bindings, covers and the like.

A printed product of paper, vinyl or other substrates including and not limited to those identified above in Example 1(e) treated by one or more of the methods of Example 23 that at least partially, preferably completely eliminated exposure to volatile organic compounds and moderately volatile organic compounds including those listed in Example 1(i) above, is provided

### EXAMPLE 24

Example 23 above, expanded to include all paper products and materials of cellulosic origin. Made and used as described in Example 23, and providing a printed and/or non-printed product of paper, and other materials of cellulosic origin that at least partially, preferably completely eliminated exposure to volatile organic compounds and moderately volatile organic compounds including those listed in Example 1 (i) above, thus preventing at least partial (preventing at least 90% VOC migration) of VOCs, of non-bound organic compounds, and the like from the interior to the surface, more preferably preventing at least 99% migration of volatile organic compounds, of non-bound organic compounds, and the like from the interior to the surface, still more preferably virtually completely free and preventing at least 99.999% migration of volatile organic compounds, of non-bound organic compounds, and the like from the interior to the surface, most preferably providing a surface completely free of migrating volatile organic compounds, of non-bound organic compounds, and the like from the interior to the surface.

### EXAMPLE 25

Paper processing and bleaching—use peroxide, similar to cloth procedure. Also use water boiling. An improved paper product is prepared having essentially eliminated small molecules, including small organic molecules. How to make: paper with reduced or eliminated volatile organic compounds (as defined in Example 1(i)) levels is obtained by selecting non-organic substances as surfactants, scouring agents, ph adjustment agents, and chelating agents, reaction control agents, and eliminating coating agents. And heating time and temperature of the art, is increased to include boiling and boiling for increase lengths of time. Although pressure allows higher temperatures to be achieved, boiling has the advantage of passing large amounts of water vapor through the reaction milieu, maximizing conditions for release of volatile organic compounds and moderately volatile organic compounds. Either boiling alone may be used or combinations of pressure and boiling. Sodium hexametaphosphate is added as a chelating agent. Then hydrogen peroxide in combination with sodium silicate is added to the bleaching step, followed by steam drying in the absence of gas dryers. Water for washes and rinses is prior purified with activated carbon and or other effective means for removal of chlorinated and nonchlorinated organic compounds. This produces an ultrapure paper product free of impurities, not previously known. Steps of gas fired drying, and organic coatings such as are eliminated. Sodium hexametaphosphate replaced sodium polyacrylate and sodium gluconate as chelating agents, and sodium silicate replaced complex phosphonate as stabilizer during the bleaching process.

How to use: this ultrapure paper is free of natural waxes and terepinoids and the like, and also free of contaminants that would otherwise be added during processing. It is useful for individuals with severe chemical sensitivities, to avoid serious, adverse reactions, and others who want to maintain good health.

Also for use with hypersensitive individuals having at least one symptom or condition selected from the group consisting of autism, anxiety, arthritis, asthma, colic, congestion, diabetes, digestive upsets, irritable bowel syndrome, eczema, fatigue, migraine headaches, multiple sclerosis, seizures and rashes, Diarrhea, constipation, congestion, eczema, asthma, fatigue, muscle weakness, tension, and spasms, irritable bowel syndrome, swelling, anxiety, multiple chemical sensitivities, moderate to extensive and moderate to severe symptoms due to food allergies, sensitivities, and intolerances, bloating, pain, headaches, leaky gut, hyperactivity, sleeping difficulties, severe underweight, eating disorders, obsessive, compulsive disorders, panic attacks, sensory sensitivities, Alzheimer's disease, acid reflux, irritability, delayed motor skills, delayed social skills, autism, PDD, infantile spasms, seizures.

The paper product free of volatile organic compounds, free of non-bound organic compounds is provided, in which the paper is preferably at least partially free (at least 90% VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like.

### EXAMPLE 26

Mineral based inks, applied with peroxide, and alternatively coated with thin glass layer when needed. How to make: mineral based inks made of pulverized iron oxides and other similar pigments are mixed with water and applied to the surface of the above described paper to form a hypoallergenic printed product that is free of volatile organic compounds and the like as described in Example 1(i) above. The paper of Example 25 above is more porous and thus able to receive the mineral based inks readily. In addition they are also combined with H₂O₂ to facilitate linking with cellulosic substrate of the paper. Finally as desired an ultrathin layer of SiO₂ is applied to the paper this assures that the ink will not rub off. How to use: use the printed paper in books, wallpaper, for decoration and all other ways paper is used, the product of Example 26 is especially useful for chronically ill individuals.

The product of Example 26 provides a printing ink and printed paper product that is free of volatile organic compounds, free of non-bound organic compounds, free of reaction products and byproducts, of partial polymerization products, of incomplete reaction products, of intermediates, of raw materials, of solvents, catalysts, and the like.

### EXAMPLE 27

Chemical bonding of mineral pigments to fabric, now possible with ultra pure more open and absorbent cotton, applied as chemical bond. How to make: select chemicals that bond to the more open and absorbent cotton and paper, to combine and bond chemically, thus providing a stable, permanent imprint and design, that does not wash out easily or smear. How to use: the pigments and cloth and paper thus made will be used for clothing, furniture fabrics, and the like and have the added advantage of providing products free of volatile organic compounds including those listed in Example 1(i) above. Mineral pigments capable of bonding with paper, cotton and other items, provide a permanent combination resistant to smearing, washing and the like, and provide a product preferably free of volatile organic compounds including those listed in Example 1(i) above, and the printed products derived from them, preferably at least partially free (at least 90 % VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like.

### EXAMPLE 28

Vegetable oil purification, carbon filtration to avoid plastic contamination or remove chemical and plastic contamination. The oil available of today has two problems, it is thoroughly contaminated with plastics and plasticizers, and it contains compounds that are volatile organic compounds and moderately volatile organic compounds including those listed in Example 1(i) above. A purification process is needed to address both issues. Oil purification includes steps of bleaching, decolorization, solvent extraction and the like. These are aimed at removal of impurities and increasing the yield of oil. Expeller pressed oils still have problems with plastic impurities and volatile organic compounds and moderately volatile organic compounds that are naturally present in the oil. How to make: to the steps conventional to the art, add a final step of filtering or slurrying and filtering using activated carbon to remove the volatile organic compounds and moderately volatile organic compounds from the oil and the plasticizers and other substances that are residues from plastic exposure. Alternately treat, rinse, swirl, bubble through H₂O₂ to bleach further, and remove the volatile organic compounds and moderately volatile organic compounds. How to use: the product thus product has wide applicability for individuals of many chronic diseases and disorders to aid in their recoveries; it will be particularly beneficial to individuals who have developed a sensitivity or intolerance to particular oils. The product of Example 28 is an oil product free of residues associated with contact with plastic materials, and free in general of volatile organic compounds and moderately volatile organic compounds including those listed in Example 1(i) above, preferably at least partially free (at least 90% VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like.

### EXAMPLE 29

Meat treatment with peroxide, not chlorine. How to make: a process for treating meat that substitutes H₂O₂ for every place in which chlorine or chlorine containing chemicals are used. Process includes H₂O₂ as liquid and as gas. How to use: use in meat processing plants, and other places that guard against infectious agents including nurseries with plant cuttings. The treatment of Example 29 produces meat products free of chlorine related contamination, yet providing a product free of infectious agents according to the conventions of the meat industry. Switching completely to H₂O₂.

### EXAMPLE 30

Peroxide in water pipes instead of chlorine. How to make: substitute peroxide addition to water and water pipes in place of chlorine. This will initially involve higher rates of use of H₂O₂, however this is due to the action of H₂O₂ on surfaces of pipes and on organic matter in the water. As water purification is increased to provide a more pure form of water, and as pipes are purified, lower levels of peroxide will be needed to achieve the desired control and quality of water. How to use: use in water treatment plants, water purification plants, wherever water purification systems are employed, add to water just prior to discharging into water pipes. A water product that provides antibiotic properties and maintains the potable characteristics of water in the absence of chlorine and chlorine containing compounds or substances, thus reducing or eliminating and avoiding volatile organic compounds including those listed in Example 1(i) above in potable water, drinking water and other purified waters, and also providing a treated water product that is preferably at least partially free (at least 90% VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like.

### EXAMPLE 31

Mattresses: stainless steel springs with or without sodium silicate coating, no oil coatings, cotton batting. How to make: mattresses free of volatile organic compounds and moderately volatile organic compounds that are appropriately comfortable, soft and the like, are prepared by obtaining bare metal springs, including stainless steel springs, that do not contain machine oil, or in which machine oil from extrusion has been removed. The metal springs, of any shape and style of the art, are coated by spraying, dipping or other desired means, with sodium silicate or other material capable of forming glassy layers, said sodium silicate or other coating is to reduce, eliminate or avoid problems associated with rust for the metal. Cotton batting as described above, free of volatile organic compounds including those listed in Example 1(i) above is used to make the rest of the mattress, applied in thickness as desired to provide appropriate support and comfort, outer cover of cotton as described above for fabric is used for the outer cover, thus providing a comfortable mattress free of volatile organic compounds and moderately volatile organic compounds a printed product of paper, vinyl or other substrates including and not limited to those identified above in Example 1(e) that at least partially, preferably completely eliminated exposure to volatile organic compounds and moderately volatile organic compounds including those listed in Example 1(i) above, and eliminating problems associated with springs, filling and cover. Similarly a mattress pad without springs is made. The instant invention of Example 31 provides a hypoallergenic mattress, futon mattress pad, and or pillow suitable for individuals with a wide variety of conditions, diseases and disorders that is free of volatile organic compounds, free of non-bound organic compounds, free of reaction products and byproducts, partial polymerization products, incomplete reaction products, intermediates, raw materials, solvents used in manufacture, catalysts, and the like that may have otherwise been present on the metal springs and other components of the matress.

### EXAMPLE 32

Elasticity, springing for cotton fibers by interior reactions inside the lumen of the fibers. Reacting sodium silicate and phosphoric acid to form silica gel in the interior. This will provide flexibility to the fibers. How to make: through the process of alternating high vacuum and high pressure, introduce first sodium silicate to the interior (lumen) of cotton fibers, then after drying similarly introduce ammonium phosphate or similar substances to form a hydrated silica gel compound in the interiors of the fibers, alternately provide the components in any desired order. The result is hydrated silica gel trapped in the interior of cotton fibers. Providing different properties, including swelling and increased spring in the fibers. This makes possible the preparation of cotton products that have spring, bounce and shape memory and that are not permanently crushed when pressure is applied. This provides improved products in bedding including mattresses and pillows among other things. How to use: using this process enhances the properties of cotton when used as bedding, pillows, cushions, furniture and the like the method of Example 32 provides an improved cotton and other cellulosic materials, with increased elasticity and resistance to crushing, while avoiding the problems of VOC exposure described elsewhere.

### EXAMPLE 33

Provide a soft bed, with spring, cushion, comfort and no adverse health effects. Make and use as described in Examples 31 and 32 above. The product of Example 33 provides a soft bed, with springs, cushion, comfort and no adverse health effects associated with exposure to volatile organic compounds and moderately volatile organic compounds including those listed in Example 1(i) above, with spring and comfort provided by fibers containing hydrated silica gel, thus preferably the bedding, mattress, pillows and other products are at least partially free (at least 90% VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like.

### EXAMPLE 34

Asphaltic shingles: spray w SiO₂ or SiO or other suitable substance as described in Example 1 (a) by vapor deposition, or alternatively apply sodium silicate to surface and heat by microwave or other methods to form and insoluble sodium silicate layer at the surface. This will eliminate the problems of release of volatile organic compounds and moderately volatile organic compounds and increase the life of the shingles and increase with water repellant properties of the shingles. How to make: problems associated with volatile organic compounds and moderately volatile organic compounds emanating from the asphaltic materials is eliminated by treating the shingles with sodium silicate. Spray w SiO₂ or SiO or other suitable substance as described in Example 1(a) by vapor deposition, or alternatively apply sodium silicate to surface and heat by microwave or other methods to form and insoluble sodium silicate layer at the surface. This will eliminate the problems of release of volatile organic compounds and moderately volatile organic compounds and increase the life of the shingles and increase with water repellant properties of the shingles. How to use: use the instant invention according to the conventions of the art. The product of Example 34 is a shingle product that has increased water repellant properties, increased useful life, and at least reduces to eliminates migration of volatile organic compounds and moderately volatile organic compounds from the shingles, at least partially free (preventing at least 90% VOC migration) of VOCs, of non-bound organic compounds, and the like, more preferably are mostly free (preventing at least 99% VOC migration) of volatile organic compounds, of non-bound organic compounds, and the like, still more preferably virtually completely free (preventing at least 99.999% VOC migration) of volatile organic compounds, of non-bound organic compounds, and the like, most preferably completely free of migrating volatile organic compounds, of non-bound organic compounds, and the like.

### EXAMPLE 35

Asphaltic shingles; mix with sodium silicate to cut down on the fumes. How to make: to the step of forming and shaping, add sodium silicate as powder or concentrated liquid to the asphaltic mass, the effect is to intermingle the sodium silicate and asphaltic, tarry mixture. Heat treatment after shingle formation will serve to capture the tarry mixture within a silicaceous mixture, thus trapping the volatile organic compounds and moderately volatile organic compounds. This process continues further after application to rooftops and exposure to heat from the sun. How to use: use the instant invention according to the conventions of the art. The resultant product is a shingle product that has increased water repellant properties, increased useful life, and at least reduces, preferably eliminates migration of volatile organic compounds and moderately volatile organic compounds from the shingles, preventing at least partial (preventing at least 90% VOC migration) of VOCs, of non-bound organic compounds, and the like, more preferably preventing at least 99% migration of volatile organic compounds, of non-bound organic compounds, and the like, still more preferably virtually completely free and preventing at least 99.999% migration of volatile organic compounds, of non-bound organic compounds, and the like, most preferably providing a surface completely free of migrating volatile organic compounds, of non-bound organic compounds, and the like.

### EXAMPLE 36

Pretreat asphalt with peroxide, O₂, O₃, air, N₂, inert gases and mixtures to eliminate the volatile organic compounds. How to make: to the step of forming and shaping, infuse the tarry mass with peroxide, O₂, O₃, air, N₂, inert gases and mixtures as required to at least reduce, preferably eliminate volatile organic compounds and moderately volatile organic compounds including those listed in Example 1(i) above the effect is to intermingle peroxide, O₂, O₃, air, N₂, inert gases and mixtures with the tarry mixture. Thus enabling contact with and removal of the volatile organic compounds and moderately volatile organic compounds and other substances identified from the matrix. How to use: use the instant invention according to the conventions of the art. The resultant product is a shingle product that at least reduces, preferably eliminates volatile organic compounds and moderately volatile organic compounds from the shingles, and at least reduces, preferably eliminates migration of volatile organic compounds and moderately volatile organic compounds from the shingles, preventing at least partial (preventing at least 90% VOC migration) of VOCs, of non-bound organic compounds, and the like, more preferably preventing at least 99% migration of volatile organic compounds, of non-bound organic compounds, and the like, still more preferably virtually completely free and preventing at least 99.999% migration of volatile organic compounds, of non-bound organic compounds, and the like, most preferably providing a surface completely free of migrating volatile organic compounds, of non-bound organic compounds, and the like.

### EXAMPLE 37

Replace tar paper underlayment with sodium silicate treated paper that is cured, or vapor deposit w SiO₂ and the like. How to make: heavy paper suitable for use as underlayment is treated with sodium silicate and cured or treated with SiO₂ and/or other substances as is described previously, above. How to use: use wherever tar paper underlayment is used. The product of Example 37 is a treated paper, resistant to moisture, with long useful life, suitable for use as underlayment and all other uses where such material is used, is most preferably free of vaporizing volatile organic compounds and moderately volatile organic compounds including those listed in Example 1(i) above, and at least reduces, preferably eliminates migration of volatile organic compounds and moderately volatile organic compounds from the shingles, preventing at least partial (preventing at least 90% VOC migration) of VOCs, of non-bound organic compounds, and the like, more preferably preventing at least 99% migration of volatile organic compounds, of non-bound organic compounds, and the like, still more preferably virtually completely free and preventing at least 99.999% migration of volatile organic compounds, of non-bound organic compounds, and the like, most preferably providing a surface completely free of migrating volatile organic compounds, of non-bound organic compounds, and the like.

### EXAMPLE 38

Insulation: binderless fiberglass w sodium silicate as binder, sodium silicate spray onto cotton or shredded paper, roll into batting; alternatively push bats in place and spray with sodium silicate to secure backing with metal foil, metallized mylar or brown paper. How to make: glass fibers of the art, suitable for use in fiberglass and insulation, are treated with sodium silicate applied as a spray. In manufacture the glass fibers are formed into batting and insulation paper is glued onto the paper, using sodium silicate or similar material as adhesive. Batting is then rolled up and used according to the conventions of the art. In addition, the batting is sprayed with sodium silicate to hold the fibers together. This occurs at any desired point in the manufacturing process. The use of the insulation, provides sufficient protection for the sodium silicate, and no heat treatment etc is needed. Alternately, use as described above with the exception that shredded cellulosic material is used in place of glass fibers. Alternatively, use foil, double sided metallized mylar, or paper described earlier in the present invention, secured with sodium silicate adhesive to the glass fiber batting to form rolls. How to use: use according to the conventions of the art and provide an insulation material free of volatile organic compounds and moderately volatile organic compounds including those listed in Example 1(i) above. The insulation material thus described is essentially free of volatile organic compounds and moderately volatile organic compounds, providing effectiveness, long useful life, and ease of use and installation.

### EXAMPLE 39

Blow in binderless insulation, spray in place w sodium silicate. Alternatively use thinner sodium silicate concentration than in Example 38 to spray in place. How to make: blow glass fibers without binders are sprayed in place, and then sprayed with sodium silicate to secure them in place. Concentration of sodium silicate solution is preferably 20% and less, more preferably 10% and less, still more preferably 5% and less, including 1% and less. How to use: use with blown insulation systems according to the conventions of the art. The resultant product is a blow in, binderless insulation with or without a final spray of sodium silicate in concentrations ranging from 20% to less than 1%, that eliminates organic materials in the product, including volatile organic compounds and moderately volatile organic compounds including those listed in Example 1(i) above.

### EXAMPLE 40

Wall paper SiO₂ vapor deposition over vinyl, inside and out. Sodium silicate for wall paper paste, vapor deposition instead of vinyl, extra heat to outgas. How to make: as described above for plastics, resins and polymers of all types, SiO₂ deposition achieves an effective barrier preventing release or migration of volatile organic compounds or moderately volatile organic compounds including those listed in Example 1 (i) above. The product thus formed and used as wallpaper is applied using sodium silicate as the adhesive replacing wallpaper paste. How to use: use as wallpaper, and use sodium silicate as replacement for wallpaper paste. The results is a wallpaper and method for installation that is free of migrating or releases of volatile organic compounds and moderately volatile organic compounds, and preferably is at least partially free (at least 90% VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like.

### EXAMPLE 41

Carpet: fibers combined with sodium silicate, polymer matrix treated with peroxide, ozone, O₂ N₂ etc to remove volatile organic compounds. Carpet pad: ultrapure cotton infused with silica gel and O₃ treated. How to make: cotton and other natural fibers treated to remove the volatile organic compounds and moderately volatile organic compounds fractions are woven into carpets. The fibers, before weaving are coated with SiO₂ or SiO and other suitable substances as identified in Example 1(a) above. The purpose is to provide a carpet of nontoxic fibers that are also resistant to moisture, spills, stains and the like. Alternatively, conventional polymer matrix described above Example 1(e) are treated with peroxide, ozone, O₂ N₂ etc to remove volatile organic compounds as described in Example 7 to reduce or eliminate the volatile organic compounds and moderately volatile organic compounds including those listed in Example 1(i) above. This is also used to prepare carpet pad materials. Alternately, a carpet pad is prepared by the process of infusing ultrapure cotton with hydrated silica gel and forming the strands into a carpet pad. How to use: use the invention as floor covering, rug, carpet and carpet pad according to the conventions of the art. Thus Example 41 provides a carpet and carpet pad at least partially to completely free of volatile organic compounds and moderately volatile organic compounds including those listed in Example 1(i) above, and preferably the resultant products of Example 41 are at least partially free (at least 90% VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like.

### EXAMPLE 42

Carpet glue: tighter weaving, spray w sodium silicate, and heat treat to cure. Use high pressure cavitation and nozzle to speed polymerization process, also microwave and radiant heat curing. How to make: spray woven carpet with sodium silicate, and heat treat to cure the sodium silicate. Use high pressure nozzle with stream impact described earlier to speed polymerization process, also may include microwave and radiant heat curing simultaneously or sequentially to increase curing speed. How to use: use to glue back of carpet to secure the fibers into place. The carpet glues of example 42 provide the desired properties while avoiding contributing to chronic health problems associated with volatile organic compounds and moderately volatile organic compounds, and preferably provide at least partially free (at least 90% VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like.

### EXAMPLE 43

Treat under home: sodium silicate layers to discourage termites, including use of impregnated paper layer. How to make: impregnated paper as described for paper underlayment is suitable for placing under the home as an avoidance barrier and treatment for termites. How to use: place under home foundation, during construction as a termite barrier and replacement for pesticides. This is possible because termites are strongly repelled by sodium silicate. The product of Example 43 is a sodium silicate impregnated paper that is strongly repellant to termites, thus eliminating the immune system triggers and the health problems associated with pesticides and associated solvents, vehicles and carriers currently placed under homes for termite control.

### EXAMPLE 44

Tyvek wrap of home: dense paper layer sprayed with sodium silicate and cured. How to make: as described in Example 37. How to use: use for wrapping home as alternative to Tyvek types of materials. The home wrap of Example 44 provides a treated paper, resistant to moisture, with long useful life, suitable for use for wrapping exterior of homes, buildings and the like and all other uses where such material is used, is free of vaporizing volatile organic compounds and moderately volatile organic compounds including those listed in Example 1(i) above, and preferably at least partially free (at least 90% VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like.

### EXAMPLE 45

Siding, vinyl: treat as for polymers, noted previously. Especially increase curing and baking time, add peroxide and ozone to curing, alternatively also mix w sodium silicate, and/or spray with sodium silicate, alternately use SiO₂ vapor deposition to coat exterior of product. Make and use as described previously for treating, mixing with, applying to, and/or applying onto the surface of plastics, polymers and resins in Examples including but not limited to Examples 1-7, 34-36. The siding product is produced to be at least partially free to completely free of volatile organic compounds and moderately volatile organic compounds including those listed in Example 1(i) above, preferably at least partially free (at least 90% VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like.

### EXAMPLE 46

Treatment for Styrofoam insulation: spray w sodium silicate and cure before use, foil wrap. How to make: spray w sodium silicate and cure before use, foil wrap. How to use: use as appropriate for Styrofoam. The resultant product isolates the VOCs inside the sodium silicate/foil barrier and effectively prevents the VOC fumes from migrating to the exterior of the product. The resultant products is a treated Styrofoam product that essentially eliminates exposure to volatile organic compounds and moderately volatile organic compounds including those listed in Example 1(i) above, by preventing them from migrating outward from the interior of the product.

### EXAMPLE 47

Mention foil wrap options a lot. Wrap composite wood products of all kinds, insulation, Styrofoam, dimension lumber, furniture, walls, wall paper, insulation, paper, toys, etc. How to make: any desired product is wrapped with foil, especially bare metal foil, most frequently aluminum foil. Applied and sealed to the wrapped item with sodium silicate. How to use: provides a method for quickly eliminating emissions of volatile organic compounds and moderately volatile organic compounds including those listed in Example 1(i) above by wrapping the object, item in bare metal foil. The instant foil wrapped products are obtained for the purpose of reducing or eliminating emissions of volatile organic compounds and moderately volatile organic compounds.

### EXAMPLE 43

Adhesive: epoxy, latex, acrylic, etc, etc, etc. switch to sodium silicate. Use animal glues, for wet applications use sodium silicate and then cure with microwave radiation, heat guns, etc. Sodium silicate as substitute for a side variety of adhesives, eliminates fully the VOC exposures and their associated immune system triggers and chronic symptoms/conditions/diseases/disorders.

### EXAMPLE 49

Caulk: whiting, titanium dioxide, various calcium compounds, etc. mix w unusual oil, let set. Also combine these w sodium silicate, mix and cure. Also use clays, of fine particle size such as kaolin and diatomaceous earth, and zeolite are combined with unusual oils and sodium silicate. How to make: caulk and similar compounds are prepared by mixing a selection of materials including but not limited to whiting, titanium dioxide, various calcium compounds, in any desired combination with a purified oil described above. How to use: The product thus formed is applied to windows as glazing materials, and used as appropriate as caulking, weather stripping, and the like. The caulk, glazing material, weather stripping material and the like thus produced is completely free of volatile organic compounds and moderately volatile organic compounds including those listed in Example 1(i) above. Also included in the example is combinations with other materials of the art in quantities and techniques that are preferably at least partially free (at least 90% VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like

### EXAMPLE 50

Air conditioner, HVAC systems. Window units were the best choice for the air conditioning task. They can be located in individual rooms and thus eliminate the need for duct work. This is important because corners and changes in surface levels inside the ductwork create small eddies and slow spaces in which particles entrained in the air flow drop out in these spaces. This creates dusty areas that are periodically stirred up and release significant amounts of dust and debris back into the room. In addition to dust, mold spores then to accumulate in these same areas. The duct work also can be a source of volatile organic compounds due to substances the ducts are composed of.

How to make: to modify an air conditioning unit suitable for cooling a few rooms, remove all plastic materials from the interior of the unit. Remove the insulation including foam and Styrofoam insulation. Remove glues, resins, adhesives, and the like from the air chamber. Cover with bake metal foil in areas that glue cannot be removed. Isolate the air cooling chamber from the remainder of the unit, by covering the entire surface of the air cooling chamber with foil using sodium silicate as an adhesive. Close and seal with bare metal foil, flashing and the like, the outside air makeup opening, unless alternatively an activated carbon filter in all metal housing is placed at the outside air makeup opening. Remove decorative and function covers from the front of the unit, either leaving the unit open to the room or providing an alternate cover for the unit of bare metal cleaned to remove any machine or mineral oils and the like, cardboard or other stiff material covered with bare metal foil, and the like.

Alternatively, prepare an all-metal container and contents having no plastics, glues, resins, adhesives or the like present in the container, free of paint, coatings, and the like, and containing no substance from Example 1 (e) above in any form, structure, function or coating in the unit, and said metal container is free of oils, waxes, coatings and the like, and/or has been cleaned with soap and/or other materials suitable removal of same with rinsing or other processes suitable and appropriate for leaving no residues themselves. Preferably said metal container is made of aluminum, stain less steel, brass, copper, gold, and the like and combinations of same, to provide an all metal container free of volatile organic compounds and moderately volatile organic compounds including those described in Example 1(i) above. Said all metal container provides a method of directly incorporating outside air without passing over or through components of the unit that contain polymers, polymer combinations and the like, including those described in Example 1(e) above, or alternatively closes and seals with metal foil and sodium silicate, the passage providing outside makeup air.

For the above air conditioning alternatives, electrical connections are preferably made with direct connection and no open wires or cords into a part of the unit that has been sealed with bare metal foil and sodium silicate so that no volatile organic compounds, moderately volatile organic compounds including those described in Example 1(i) above, are able to migrate into the air cooling chamber, or alternatively the cords, wires etc present in the area cooled by the air conditioner unit are made of non volatile organic compounds, non moderately volatile organic compounds including those described in Example 1(i) above or alternatively are covered with bare metal foil using sodium silicate as an adhesive.

Larger air conditioning units are also provided made as described above. In addition the systems additional components, including ductwork, vents, fans, and all air handling components that bring in air, cool or heat air, and convey air to areas inhabited by individuals, follow the above described criteria for providing pure metal materials, or materials that have been covered with bare metal foil sealed with and kept in place with sodium silicate. Makeup outside air similarly passes through pure metal materials, or materials that have been covered with bare metal foil sealed with and kept in place with sodium silicate. Connectors for ductwork shall be made of metal or shall be wrapped completely themselves in bare metal foil sealed with and kept in place with sodium silicate. Said outside makeup air optionally includes an activated carbon filter that is contained within al all metal housing, said filter to remove volatile organic compounds and moderately volatile organic compounds including those described in Example 1(i) above when outside air is not sufficiently pristine or when said air passes through or near polymers or polymer mixtures including those described in Example 1 (e) above.

Alternately said air heating and cooling systems made for conventional materials, provide for air purification at each air outlet, said filter to include a HEPA filter substitute described below for particulate removal, and an activated carbon air filter, each assisted as necessary by an all metal booster fan located prefilter.

HEPA filter substitute that avoids adhesives and resins of the HEPA filter, includes filtering fibers of appropriately sized glass fibers arranged in the desired way, to which is applied a spray of sodium silicate. Said sodium silicate of sufficient concentration to coat and seal in place the fibers while providing the desired particle size openings in the filter. Alternatively the sodium silicate is provided in a means that provides uniform coverage of the glass fibers leaving no holes and then while still wet, either with air puffs through a grid, mechanical means, or other techniques of the art, holes of the desired size are created in the matrix of glass and sodium silicate. The sodium silicate is then polymerized to insoluble form of sodium silicate by techniques described elsewhere in the present application, thus providing a matrix that completely captures and holds in place the glass fibers and provides a consistent, repeatable filter openings for passage of air.

Such filter provides effective filtration equivalent to HEPA filters without also providing exposure to volatile organic compounds or moderately volatile organic compounds including those described in Example 1(i) above that may be contained on the filter or filtration matrix. Invention is suitable for filtration of any desired particle size and mesh openings, including those not in the range of a HEPA filter. Said filter also capable of being combined with other substances in addition to glass fibers, including and not limited to metal fibers, cellulosic fibers, and the like, and also alternatively to include a wide variety of resins and filter materials including those described in Example 1(e) above to which a thin coating of sodium silicate is applied and then converted to insoluble form of sodium silicate as described previously in the present application thus providing filters of the desired opening without also providing exposure to volatile organic compounds and moderately volatile organic compounds including those described in Example 1(i) above.

How to use: use as an air conditioner in ways that are the conventions of the art. Use as substitute for HEPA filter and for other filters of the art.

Included in Example 50 are air conditioners, air conditioning systems, air heating and air conditioning systems, air filters and the like that provide the desired functions while at least partially and preferably totally eliminate exposure to volatile organic compounds and moderately volatile organic compounds; preferably surfaces exposed directly or indirectly to the air stream and the air emanating from vents, and discharge points is at least partially free (at least 90% VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like.

### EXAMPLE 51

Water filters housing. How to make: housing for water filters is provided that is free of polymers and polymer mixtures either by manufacture from metal, glass, and other suitable materials or by use of the above described materials that have been modified to at least partially, preferably at least mostly, more preferably substantially, most preferably completely eliminate migration of volatile organic compounds and moderately volatile organic compounds from the housing. How to use: said filter housing available for use according to the conventions of the art. The filter housings and canisters of the instant Example provide water filters and air filters that are partially to completely free of or prevent migration either partially or completely of, volatile organic compounds and moderately volatile organic compounds, preferably at least partially free (at least 90% VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like.

### EXAMPLE 52

Water filter insert. How to make: an activated carbon filter or filter of other appropriate material that at least partially or completely eliminated or prevents volatile organic compounds or moderately volatile organic compounds from passing through and or emanating from the filter media, said filter also to have no components derived from polymers or polymer mixtures, or other materials described elsewhere in the present application including cellulosic materials and other biological materials unless the above described methods for reducing or eliminating volatile organic compounds or moderately volatile organic compounds emissions from said materials has been employed or applied. Thus the filter media, filter housing, and all materials for providing water seals, adhesives and the like provide a product free of moderately volatile organic compounds and volatile organic compounds, providing effective filtering and providing no contributing substances to the water stream from the product. Similarly air filters including air filters for insertion into windows can be made by the above described method. Similarly personal filters can be made by the above described method. How to use: us the water filters according to the conventions of the art.

As example, a water filter is provided with housing and connectors of stainless steel, aluminum and brass, into which is placed a filter bag of cotton treated according to the present application, containing activated carbon of suitable particle size. Thus providing filter housing, filter media, and filter bag free of volatile organic compounds and moderately volatile organic compounds, capable of effective filtration of the art. Thus is produced a water filter free of or at least partially free of volatile organic compounds and moderately volatile organic compounds, also produced are air filters free of or at least partially free of volatile organic compounds and moderately volatile organic compounds, in which preferably all varieties of air and water filters are at least partially free (at least 90% VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like.

### EXAMPLE 53

Water distillers. How to make: a water distiller is made providing all metal or glass construction, all metal or glass connectors, tubing and conveyance, and all collecting equipment of glass or metal, and all incoming water lines filtered with the above described instant filters. Or alternatively any polymers or polymer mixtures or other nonmetal and nonglass components are treated as described above in the present application so that the emission of, migration of, presence of volatile organic compounds and moderately volatile organic compounds is at least partially, preferably mostly, still more preferably substantially all, most preferably completely all eliminated. How to use: a water distiller providing a water distilled product free of volatile organic compounds and moderately volatile organic compounds that may be present either in the distilled product or introduced as a contaminant through contact with polymers and polymer mixtures in materials of construction, conveyance, containment, and the like. The resultant water distiller and distillation products are partially or completely free of volatile organic compounds and moderately volatile organic compounds, preferably at least partially free (at least 90% VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like.

### EXAMPLE 54

Soap, lotions, lip balms, baked goods, emollients using the oils. How to make: make soap, lotions lip balms, baked goods, emollients, and the like, according to the conventions of the art, with the addition of selecting the purified oils described previously in the present application for use in the above products and any other product known or described in the art.

How to use: use the products thus obtained according to the conventions of the art. Improved products of soap, lotions lip balms, baked goods, emollients, and the like are provided because they are at least partially to completely free of volatile organic compounds and moderately volatile organic compounds otherwise present in the oils.

### EXAMPLE 55

Mask, cotton. How to make: a cotton face mask is made from the cotton of the present invention. In one alternative the face mask design is such that the mask fits tightly around the nose and mouth. How to use: use to make masks of the cotton of the present invention. A face mask of cotton that is at least partially to completely free of volatile organic compounds and moderately volatile organic compounds is provided, that also fits tightly to the face and avoids leakage around stitches.

### EXAMPLE 56

Cotton mask insert. How to make: an insert comprised of cotton of the present invention and activated carbon, including and not limited to the following: small particles of carbon approximately 1 mm in diameter and less that are interspersed among teased apart cotton fibers until they fall among the fibers to form a cotton fiber—carbon matrix having combined properties of both and in which the carbon, held by and enmeshed in the fibers does not migrate from the insert material. Ultrapure Cotton fabric of the present invention is then added and tacked in places to form a cotton pad, or insert. Said insert having strength, and capability to be molded into and hold desired shapes, allowing said insert to conform to the shape of mouth, nose and chin thus providing a tight fit and effective seal and filtration capability. How to use: use with cotton mask to provide effective filtration of air without providing added volatile organic compounds and moderately volatile organic compounds from the mask and filter materials. The resultant products thus produced are ultrapure cotton mask inserts that provide the desired air purification and at least partially to completely eliminate volatile organic compounds and moderately volatile organic compound-related emissions from materials of construction. Also cotton masks and inserts of a wide variety of designs and configurations that provide the desired air purification and at least partially to completely eliminates volatile organic compounds and moderately volatile organic compounds emissions from materials of construction.

### EXAMPLE 57

Metallized mylar cap, scarf, top, pants, and clothing, and throw cover, car, back seat, front seat How to make: clothing may be sewn of metallized mylar, that has metal coating deposited on both sides of the mylar, similarly any desired polymer or polymer mixture may be similarly coated on both sides with a thin layer of deposited metal, provided according to the conventions of the art. This is included in the use of the term metallized mylar, in the present invention. Metallized mylar coated on both sides has the advantage of effectively sealing within the inside matrix all residues, volatile organic compounds and moderately volatile organic compounds present in the polymer or polymer mixture and effectively preventing their migration from the polymer and polymer matrix. At the same time, metallized mylar provides an effective vapor barrier similarly preventing migration of vapors associated with anything it is placed on top of or wrapped around. Hence clothing including and not limited to hats, scarves, pants, tops, robes, gowns, diaper covers and diaper cover inserts and the like may be made from double sided metallized mylar, with adjustments, and suitably tight fits around head, neck, wrists, waist, ankles and the like, and prevent any residues underneath the garment from migrating through the double side metal coated metallized mylar to the outside. This makes such garments effective barriers that can greatly protect very ill people when they are worm by the persons who care for them. Double sided metallized mylar and similar materials described above may be used separately or together with ultrapure cotton of the instant invention to provide throw covers, including for cars, back seat, front seat. How to use: for use by caregivers and other including teachers and visitors who are near enough to an ill person to cause effects from volatile organic compounds and moderately volatile organic compounds present in hair and body surfaces. The metallized mylar clothing described herein provides a hypoallergenic clothing barrier for ill or sensitive people that is comprised of a double sided coating of metal over a layer of polymer or polymer mixture providing flexibility of the polymer and effective vapor barrier of the metal, it is a way for individuals who may be wearing clothing containing VOCs or whose body is contaminated in various ways for various reasons to eliminate VOC exposure to other individuals. Double coated mylar and other polymers and polymer mixtures coated on both sides with thin deposits of metal forming an effective vapor barrier, thus preventing migration of volatile organic compounds and moderately volatile organic compounds either through the material or from the material, capable of the above described and many other uses.

### EXAMPLE 58

Pollen protection garment

How to make: a light weight ultrapure cotton garment of the present invention that covers clothing and prevent pollen from contacting clothing, includes garments and hats and hair coverings.

How to use: for use during pollen season or dust storms.

Claim: an ultrapure cotton garment covering hair and/or clothing to prevent pollen or dust contact with hair or clothing.

### EXAMPLE 59

Door seal, top, side. How to make: seals for doors, top sides and floor, and seals for windows, and seals for any area that involves cracks and gaps may be made from ultrapure cotton fo the present invention sew into shapes of long tubes and filled with activated carbon, forming effective filters for gaps and cracks. How to use: use to prevent volatile organic compounds and moderately volatile organic compounds from entering a room or space through cracks and gaps in doors, windows, closets, walls and the like. The resultant products of Example 59 provide filters and seals that prevent volatile organic compounds and moderately volatile organic compounds from entering a room or space through cracks and gaps in doors, windows, closets, walls and the like, preferably the result is filters and seals that provide treated air that is at least partially free (at least 90% VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like.

### EXAMPLE 60

metal heater. How to make: a heater of metal, including an oil filled metal core heater that is free of paint and any other coating including coatings of oil of coatings of polymers and/or polymer mixtures. Make at time of manufacture or make by sanding blasting or other techniques that remove said coatings without adding new moderately volatile organic compounds and moderately volatile organic compounds, said removal provided for all components and parts so that no emissions of volatile organic compounds or moderately volatile organic compounds occur. How to use: use according to conventions of the art. The product of Example 60 is a variety of heaters free of volatile organic compounds and moderately volatile organic compounds emissions suitable for use with ill or very sensitive individuals, preferably at least partially free (at least 90% VOC free) of volatile organic compounds, free of non-bound organic compounds, free of reaction products and byproducts, partial polymerization products, incomplete reaction products, intermediates, raw materials, solvents used in manufacture, catalysts, and the like, more preferably are mostly free (at least 99% VOC free) of volatile organic compounds, of non-bound organic compounds, of reaction products and byproducts, partial polymerization products, incomplete reaction products, intermediates, raw materials, solvents used in manufacture, catalysts, and the like, still more preferably virtually completely free (at least 99.999% VOC free) of volatile organic compounds, free of non-bound organic compounds, free of reaction products and byproducts, partial polymerization products, incomplete reaction products, intermediates, raw materials, solvents used in manufacture, catalysts, and the like, most preferably completely free of volatile organic compounds, free of non-bound organic compounds, free of reaction products and byproducts, partial polymerization products, incomplete reaction products, intermediates, raw materials, solvents used in manufacture, catalysts, and the like

Throughout this application the use of the term sodium silicate, refers to sodium silicate and all possible similar materials including those described in Example 1(a and b)

Throughout this application the use of the term polymer and polymer mixtures, refers to all possible similar materials including those described in Example 1(e and f)

Throughout this application the use of the term volatile organic compounds and moderately volatile organic compounds, refers to all possible similar materials including those described in Example 1(i, j and l)

Throughout this application the term at least partially to completely, refers to at least partially, at least 25%, preferably mostly, ie, 50% or more, preferably substantially, ie, 75% or more, more preferably essentially all, ie, 90% or more, preferably all, ie, 100%.

Throughout this application the use of the term application or applying sodium silicate as broadly defined above, refers to methods of application and all possible similar materials including those described in Example 1(c).

Throughout this application the use of the term sodium silicate, refers to sodium silicate and all possible similar materials including those described in Example 1(a and b)

Throughout this application the use of the term seal, migration, vaporization and the like, refers to terms of Example 1(g) lines 2-4.

The invention described herein reflects the inventor's discoveries and new understanding that exposure to volatile organic compounds and moderately volatile organic compounds as described in the present application are the underlying cause of symptoms/conditions/diseases/disorders, and are the substances that trigger immune system response and enzyme impairment resulting in most if not virtually all chronic diseases, disorders and conditions, and further that in the absence of exposure to these volatile organic compounds and moderately volatile organic compounds as defined herein, these chronic diseases, disorders and conditions do not occur and will not occur. Therefore modifying materials of common use in homes, businesses and the like to remove said volatile organic compounds and moderately volatile organic compounds is imperative in order to alleviate the pain and suffering caused by these chronic diseases, disorders and conditions, provide and extend healthy useful lives, and eliminate the economic losses associated with chronic diseases, disorders and conditions as they occur today.

Exemplary case studies are provided in the appendix to illustrate how various chronic conditions responded to the combined approach of dietary intervention with the Special Foods Diet, and rigorous elimination of environmental exposures and to illustrate the correlation between elimination of exposure to VOCs and elimination of chronic symptoms, conditions, diseases, and disorders.

### Appendix: Exemplary Case Studies

Exemplary case studies are attached as an appendix to illustrate how various chronic conditions responded to the combined approach of dietary intervention with the Special Foods Diet, and rigorous elimination of environmental exposures and the correlation with elimination of chronic symptoms, conditions, diseases, and disorders. Figure titles are as follows:

[image]

CASE 1. Diarrhea, Congestion, Throat Tightening, Fatigue. A 49-yo female, psychology professor, who entered the program had suffered from debilitating symptoms for three years. She had reactions to virtually all foods as well as items such as perfumes and petroleum; she also suffered from congestion, severe diarrhea, frequent throat tightening and severe fatigue. Her strong reactions had required frequent hospitalizations. FIG 1 shows the drop in symptoms that occured as dietary changes were made and as environmental exposures were eliminated.

[image]

CASE 2. PDD, Low Muscle Tone, Autism, Pica, Tantrums. 3 year old female, suffered from autism and developmental disabilities associated with low muscle tone, severe tantrums, and self-stimulating behaviors. She was anti-social with her parents and other children her age. She ate inappropriate materials including: sand, dirt, carpet fuzz, and dust pills. She had begun to react to virtually all foods prior to beginning treatment with the Special Foods Diet and elimination of VOC's exposures, the child's progress is shown in FIG. 2. After a year her social interactions, cognitive abilities, and muscle tone were normal. This child, being much less severe than many was able to fully recover within a year, by then attending a mainstream classroom without an aide, without diet restrictions, and has achieved a full recovery.

[image]

CASE 3. Severe Asthma, Hyperactivity, Poor Balance. An 8-year old female, suffered from severe asthma, occasionally requiring hospitalization, hyperactivity, attention deficit, lack of focus,,poor coordination and strong reactions to many foods and molds with hives and throat tightening. The family and child were required to simultaneously implement the extensive strategies for avoiding chemical exposures as well as complete change to the Special Foods Diet. Essentially simultaneously with these changes, her symptoms were eliminated. At the six-week point of the program, nebulizer treatments were no longer required; her symptoms were eliminated and have been maintained, as shown in FIG. 3.

[image]

CASE 4. Severe Eczema, Hyperactivity. A young male, 13 months of age, had been suffering from severe eczema covering as much as 75% of his total body surface, since age 2 months, without responding to treatment. Symptoms included swollen, puffy eyes, bright red, raw sores spreading from his mouth and chin up to both of his eyes and over much of his body. The child also had a poor appetite, poor sleep patterns and experienced constant pain.

Both nursing mom and child were placed on the Special Foods Diet and followed rigorous requirements for avoiding VOC's exposures. FIG. 4 shows the drop in symptom levels that followed the dietary and environmental changes. The child was found to react severely to VOCs as well as foods. The child has suffered no recurrence of his condition.

[image]

CASE 5. ADD, Bipolar Disorder II, Fatigue, Mind Fog. A 24 yo female entered the program experiencing worsening symptoms that unaffected by her medications, including mental sluggishness, poor memory, high error rate, frustration, feeling overwhelmed, difficulty following instructions or sequences, and experiencing emotional extremes that included frequent crying spells. FIG. 5, presents the results with the combined approach of the Special Foods Diet and elimination of VOC exposures. FIG. 5 also shows a spike in symptom levels associated with an exposure to computers and monitors.

[image]

CASE 6. Brain Stem Seizures, Low Muscle Tone, Diarrhea, Screaming. A 27 month old female entered program suffering from 15-20 brain stem seizures daily, severe diarrhea, severe diaper rash, constant spitting up, frequent vomiting of foods, low muscle tone, particularly on her right side, sensitivity to touch, light and sound, difficulty sleeping. The child screamed constantly, engaged in fast head shaking and grabbed at her face. She was also unable to show any emotion. She was entered into the program to help her become more comfortable and in less pain.

FIG. 6 shows that during the program of dietary intervention with the Special Foods Diet and removal of VOC exposures, the child experienced a gradual drop in symptoms, to the point of very low levels after one year. After 6 months, the parents noticed that the child was no longer experiencing seizures, and gradually tapered the child off anti-seizure medications over the next 6 months. The child remained seizure free.

I claim:

1. A method of increasing the effectiveness of the human immune system comprising reducing the exposure of a human patient to volatile organic compounds by locating sources of VOCs to which the human patient is being exposed and reducing the VOC exposure by coating the VOC source with a material comprising glass or a glass precursor.

2. The method of claim 1, wherein the source of the VOCs is painted surfaces, and the painted surface is coated with a glass or glass precursor.

3. The method of claim 1, wherein the source of the VOCs is the interior of a food container and the interior of the food container is coated with a glass or glass precursor.

4. The method of claim 1 wherein the source of the VOCs is an intravenous tube, and the interior of the tube is coated with a layer of glass or a glass precursor in an amount sufficient to prevent the passage of VOCs but thin enough to maintain flexibility in said tube.

5. The method of claim 1 wherein the source of the VOCs is a polymeric material, and the polymeric material is contacted with at least one material selected from the group of hydrogen peroxide, ozone, oxygen, nitrogen, and excess air prior to, or subsequent to shaping the polymeric material.

6. The method of claim 1, wherein the source of VOCs is a polymeric material and the polymeric material is pulverized into ultrfine particles passing through at least a 400 mesh screen to remove small molecules and VOCs.

7. The method of claim 1, wherein the source of VOCs is plastic food wrap and the food wrap is coated or mixed with silicon oxide or silicon dioxide.

8. The method of claim 1 wherein the source of VOCs is at least one of the structures consisting of the interior walls, floor and ceiling of an existing room and the source is spray coated with a portable vapor deposition apparatus.

9. The method of claim 1 wherein the source of VOCs is comprised of cotton and the cotton is first wetted with a surfactant, rinsed thoroughly, and sodium hydroxide and the hydrogen peroxide optionally with sodium silicate.

10. The method of claim 9 wherein the cotton is woven into fabric in looms free of VOC contamination.

11. The method of claim 9 wherein the cotton is formed into fabric by hydroentangling using water free of VOCs.

12. A method of restoring health to a patient comprising eliminating from the patient's environment sources of exposure to VOCs.

13. The method of claim 12 wherein the patient is confined to a clean room free of VOCs until his health is restored.

14. The method of claim 13 wherein the clean room is maintained in at least one of a hospital, nursing home, convalescent center, retirement home and residence.

15. A method of preventing chronic disease in a human patient comprising reducing or eliminating the exposure of the patient to VOCs.

16. A paint useful in the method of claim 15 comprising a water or H2O2 as the vehicle, unusual flours or inorganic substances as a filler and a binder.

17. The paint of claim 16 wherein the binder is a cooked flour.

18. The method of claim 15 wherein a patient's clothing is contacted with hydrogen peroxide before the clothing is donned.

19. The method of claim 15 wherein meat is treated with hydrogen peroxide prior to being ingested by a patient.

20. The method of claim 15 wherein water is contacted with hydrogen peroxide prior to being ingested by a patient.

21. The method of claim 15 wherein a mattress is formed of bed springs comprising stainless steel free of oils or VOCs emitting substances, and comprising a cotton batting formed by the process of claim 9.

22. The method of claim 15 wherein asphaltic shingles are covered by vapor deposited glass of a solution of sodium silicate which has been heat treated to form a glass.

23. The method of claim 15 wherein asphaltic shingle forming material is mixed with sodium silicate prior to shaping the shingle.

24. The method of claim 15 wherein asphalt is treated with at least one material selected from the group consisting of hydrogen peroxide, oxygen, ozone, nitrogen and inert gas in an amount sufficient to reduce VOCs emissions and formed into a shingle.

25. The method of claim 15 wherein tar paper is covered with vapor deposited glass or sodium silicate.

26. The method of claim 15 wherein insulation is blown into place and sodium silicate is contacted with said insulation.

27. The method of claim 15 wherein wallpaper comprising a polymer is coated with vapor deposited silicon dioxide.

28. The method of claim 15 wherein carpet fibers are combined with sodium silicate and treated with at least one material selected from the group comprising hydrogen peroxide, oxygen, ozone and nitrogen in an amount sufficient to remove VOCs.

29. The method of claim 15 wherein a sodium silicate treated paper is placed as an underlayment under a residence.

30. The method of claim 15 wherein a residence is wrapped with a plastic or plastic containing paper, which plastic or paper has been coated with sodium silicate.

31. The method of claim 15 wherein a foamed insulation is sprayed with sodium silicate.
